(19) United States
(12) Patent Application Publication
(10) Pub. No.: US 2013/0041348 A1
(43) Pub. Date: Feb. 14, 2013

(54) Catheter-Based Delivery of Skeletal Myoblasts to the Myocardium of Damaged Hearts

(71) Applicants: Douglas B. Jacoby, Wellesley, MA (US); Jonathan H. Dinsmore, Brookline, MA (US)

(72) Inventors: Douglas B. Jacoby, Wellesley, MA (US); Jonathan H. Dinsmore, Brookline, MA (US)

(21) Appl. No.: 13/587,332

(22) Filed: Aug. 16, 2012

Related U.S. Application Data
(63) Continuation of application No. 11/367,628, filed on Mar. 3, 2006, now abandoned.
(60) Provisional application No. 60/658,887, filed on Mar. 4, 2005.

(51) Int. Cl. A61K 35/54; A61M 25/00; A61P 9/00

(52) U.S. Cl. 604/508; 424/93.7

(57) ABSTRACT

The present invention provides improved systems and methods for the minimally invasive treatment of heart tissue deficiency, damage and/or loss, especially in patients suffering from disorders characterized by insufficient cardiac function, such as congestive heart failure or myocardial infarction. In certain embodiments, a cell composition comprising autologous skeletal myoblasts and, optionally, fibroblasts, cardiomyocytes and/or stem cells, is delivered to a subject's myocardium at or near the site of tissue deficiency, damage or loss, using an intravascular catheter with a deployable needle. Preferably, the cell transplantation is performed after identifying a region of the subject's myocardium in need of treatment. The inventive procedure, which can be repeated several times over time, results in improved structural and/or functional properties of the region treated, as well as in improved overall cardiac function. In particular, the inventive therapeutic methods may be performed on patients that have previously undergone CABG or LVAD implantation.

### RELATED APPLICATIONS

The present invention claims priority to Provisional Application No. 60/658,887 filed on Mar. 4, 2005 and entitled “Catheter-Based Delivery of Skeletal Myoblasts to the Myocardium of Damaged Hearts. The Provisional application is incorporated herein by reference in its entirety.

### BACKGROUND OF THE INVENTION

Cardiac diseases are responsible for a preponderance of health problems in the majority of industrialized countries as well as in many developing countries. In the United States, heart disease is the first leading cause of mortality, accounting for nearly 40% of all deaths (Heart and Stroke Statistical Update, American Heart Association 2002). About 85% to 90% of cardiac-related deaths are associated with ischemic heart disease, valvular disease, congenital heart disease, hypertensive heart disease and/or pulmonary hypertensive heart disease. In particular, ischemic heart disease, in its various forms, accounts for about 60-75% of all deaths caused by heart disease. One of the factors that renders ischemic heart disease so devastating is the inability (or weak capacity) of cardiac muscle cells to divide and repopulate damaged areas of the heart, making any cardiac cell loss irreversible. When they do not lead to death, cardiac diseases may result in substantial disability and loss of productivity. About 61 million Americans (almost one-fourth of the population) live with heart disorders, such as coronary heart disease, congenital heart defects, and congestive heart failure. In 2001, 298.2 billion dollars were spent in the treatment of these clinical conditions, and their economic impact on the U.S. health care system is expected to grow as the population ages.

Over the past 30 years, advances in the treatment and prevention of cardiac diseases have led to continually declining morbidity and mortality rates. Treatments for both congenital heart defects and cardiomyopathies have become more and more sophisticated. However, when these treatments fail, organ or tissue replacement remains the only other possible option. Different surgical procedures may be performed to treat heart failure and cardiac deficiency. These procedures include transplantation of organs from one individual to another, reconstructive surgery, and implantation of mechanical devices such as biventricular pacemakers or mechanical heart valves.

Cardiac transplantation is so common that the primary limitation on patient outcome is not the surgical technique, but the scarcity of suitable donor organs. In 2000, 2,500 heart transplants were performed in the U.S. while between 20,000 and 40,000 patients could have benefited from such a medical procedure. Surgical reconstruction, whereby damaged or defective tissue at one site of the patient is replaced by healthy tissue from another part of the patient's body, can help circumvent the problem of low donor organ availability. These autografts include blood vessel grafts for heart bypass surgeries. The disadvantages of using autografts are their limited durability (E. Braunwald, in: “Heart Disease”, 4^{th} Ed., E. Braunwald (Ed.), 1992, W.B. Saunders: Philadelphia, Pa., pp. 1007-1077) and a loss of function at the donor site. In addition, reconstructive surgery often involves using the body's tissues for purposes not originally intended, which can result in long-term complications. Mechanical heart valve prostheses have proved to effectively improve patient's quality of life. However, since these mechanical valve substitutes are nonviable, they have no potential to grow, to repair or to remodel; therefore their durability is limited, especially in growing children (J. E. Mayer Jr., Semin. Thorac. Cardiovasc. Surg., 1995, 7: 130-132).

Since currently available treatments (with the exception of cardiac transplantation) are only palliative, new systems and procedures for treating heart diseases, especially approaches for the recovery of diminished cardiac function, are highly desirable.

Cellular transplantation has been the focus of recent research into new means of repairing cardiac tissue, for example, after myocardial infarction. A major problem with transplantation of adult cardiac myocytes is that they do not proliferate in culture (P. D. Yoon et al., Tex. Heart Inst. J., 1995, 22: 119-125). To overcome this problem, attention has focused on the possible use of skeletal myoblasts as skeletal muscle tissue contains satellite cells which are capable of proliferation. Myoblast transplantation appears as a promising new treatment for patients with congestive heart failure and/or myocardial infarction. Successful autologous skeletal myoblast transplantation to the myocardium has been demonstrated in a variety of animal models (D. A. Taylor et al., Nature Med., 1998, 4: 929-933; B. Z. Atkins et al., Ann. Thorac. Surg., 1999, 67: 124-129; N. Dib et al., J. Endovasc. Ther., 2000, 9: 313-319), where survival and engraftment of the injected myoblasts were verified by the presence of labeled skeletal cells and multinucleated myotubes characteristic of skeletal muscle in myocardial tissue (B. Z. Atkins et al., Ann. Thorac. Surg., 1999, 67: 124-129; N. Dib et al., J. Endovasc. Ther., 2002, 9: 313-319). After injection into damaged myocardium, skeletal myoblasts were found to differentiate and develop into striated myofibers, becoming integrated into the scar tissue (D. A. Taylor et al., Nature Med., 1998, 4: 929-933).

While these methods of transplantation of skeletal myoblasts to the injured myocardium have produced promising results, they require open-heart surgery, i.e., a highly invasive procedure. Therefore, there is a clear need for alternative strategies for treating heart diseases. In particular, systems that allow heart tissue damage to be reversed or heart tissue defect to be repaired without presenting the risks and potential complications associated with general anesthesia and heart surgery are highly desirable.

### SUMMARY OF THE INVENTION

The present invention provides systems and methods that allow for the treatment of damaged/defective heart tissue, especially in individual suffering from disorders characterized by insufficient cardiac function, such as congestive heart failure or myocardial infarction. The inventive methods of treatment are simple, minimally invasive, and do not require general anesthesia or major surgical procedures. More specifically, in these methods, a cell composition is delivered to a patient's myocardium at or near the site of tissue damage using a catheter inserted into the patient's venous system. The cell transformation may be performed after identifying a region of the patient's myocardium in need of treatment. The cell compositions used in the transplantations of the invention comprise cells that are preferably isolated from the future recipient, thus avoiding tissue rejection problems. In certain embodiments, the cell composition comprises skeletal myoblasts. In other embodiments, the cell composition comprises different types of cells selected from the group consisting of skeletal myoblasts, cardiomyocytes, fibroblasts, and stem cells.

A patient may receive only one cell transplantation according to the present invention for a single dosing. Alternatively, a patient may receive multiple cell transplantations at different time points for repeated dosing. Catheter-based delivery of cell compositions according to the present invention may be performed on an individual receiving medication or other treatment for cardiac dysfunction and/or its symptoms. Alternatively, it may be performed on an individual that is not receiving any other medication or treatment for cardiac dysfunction.

In some embodiments, cell transplantation according to the present invention is performed on an individual that has previously undergone coronary artery bypass grafting (CABG) or left ventricular assist device (LVAD) implantation. The CABG or LVAD implantation procedure undergone by the patient may or may not have been accompanied by simultaneous cell transplantation. In some embodiments, cell transplantation according to the present invention is performed on an individual that is undergoing CABG or LVAD implantation (i.e., the cell composition is delivered at the time of open-chest procedure using a catheter inserted into the patient's venous system). In some embodiments, cell transplantation according to the present invention is performed on an individual that has not received and/or will not be receiving any therapy for treating damage/defective heart tissue.

### BRIEF DESCRIPTION OF THE DRAWING

FIG. 1 is a set of pictures showing that six weeks after autologous skeletal myoblast (ASM) injection in sheep with ischemic HF, composite Trichrome (A) and skeletal muscle specific myosin heavy chain (B) (MY-32, purple staining) stained sections demonstrate extensive patches of ASM-derived skeletal muscle fibers engrafted in areas of myocardial scar. In panels (C) and (D), at higher magnification from panel (A) (arrow), skeletal fibers were seen aligned with each other and further organized into myofibril bundles (Panels (C) and (D)). ASM-derived skeletal muscle aligned with remaining cardiac myocytes (Panel (E), ‘c’) and with neighboring skeletal myofibers confirmed with staining for MY-32 (F). Scale bars in panels (B), (D) and (F) are 2 mm, 0.5 mm and 0.2 mm, respectively.

FIG. 2 is a set of pictures showing that viable muscle within an area of myocardial fibrosis and scar is seen with Trichrome staining (A). Staining with MY-32 (B) confirmed that ASM-derived skeletal muscle engrafted in close proximity and aligned with remaining cardiac myocytes (‘c’)—did not selectively stain for tropinin-I (C). At higher magnification from the same area (C, arrow), ASM-derived skeletal myocytes do not stain for connexin43 (D) despite very close apposition to remaining cardiac myocytes (‘c’). Scale bars in panels A and D are 0.2 mm and 0.1 mm.

FIG. 3 shows left ventricular volume (LVV) and pressure (LVP) tracings from a single sheep before and after microembolization (top and middle panels); highlight changes in the ESPVR (middle) and the PRSW (bottom, squares) with or without ASM transplantation (bottom panel, circles) after microembolization. Though ASM transplantation did not improve cardiac function (slope) after week 1 (∘ and □), transplantation did prevent a rightward shift in the PRSW seen in the HF control animal at week six ( and ▪).

FIG. 4 shows that the left ventricular dilatation (ESVI, top panel) and the increase in mid papillary short-axis length (SA, middle panel) were attenuated after ASM injection (N=5, open bars) as compared to heart failure controls (N=6, shaded bars). Left ventricular long-axis length (LA, bottom panel) was not different between groups. All animals, including HF controls (“none”), were used to evaluate the relationship of ASM-derived myocyte survival (log of surviving cells) to that of LV remodeling (inset each panel, N=11). Animals with the highest ASM-derived myocyte survival demonstrated the greatest attenuation, particularly in LV short-axis dilatation. Correlative statistics presented for each relationship.

FIG. 5 shows results of 3-dimensional NOGA unipolar endocardial voltage mapping at transplantation/injection (A, D), sacrifice (D, E), and gross pathology of hearts at harvest (C, F). A representative control animal is shown in the top row and an animal injected with 600 million cells in the bottom row. Black dots in A and D indicate the sites of injection within the left ventricle and septal wall of heart. A color scale is shown in the upper right corner of each NOGA map with an upper and lower limit of 15 mV and 7 mV, respectively.

Table 1 presents cardiac hemodynamics in sheep after autologous skeletal myoblast transplantation as described in Example 1.

Table 2 presents left ventricular regions and segmental function data measured in sheep after autologous skeletal myoblast transplantation in sheep as described in Example 1.

Table 3 presents the design of a study aimed at demonstrating the safety and feasibility of percutaneous autologous skeletal myoblast transplantation in the coil-infarcted swine myocardium, as reported in Example 3.

Table 4 presents the retention of myoblasts in different tissues 2 hours following catheter-based injection into the myocardium of swine, as reported in Example 3.

Table 5 describes skeletal myoblast cell and dosing characteristics used percutaneous autologous skeletal autologous skeletal myoblast transplantation in the coil-infarcted swine myocardium, as reported in Example 3.

Table 6 shows cardiac functional parameters at the time of autologous skeletal myoblast transplantation (baseline) and 60 days later (sacrifice) in swine (see Example 3).

FIG. 6 is a graph showing the cumulative patient enrollment in CABG and Cell Transplantation Group.

Table 9 shows the baseline demographics in the CABG and Cell Transplantation Group of patients.

Table 10 lists the surgical procedures that the patients in the CABG and Cell Transplantation Group had underwent.

FIG. 7(A) is a graph showing the results of a flow cytometry analysis of myoblasts to be injected. FIG. 7(B-D) is a set of pictures showing myoblasts in culture (fusion is indicated by an arrow).

FIG. 8 shows NYHA Class pre and post myoblast transplantation in the CABG and Cell Transplantation Group of patients.

FIG. 9 shows electrocardiogram results (presented as ejection fraction) pre and post myoblast transplantation in the CABG and Cell Transplantation Group of patients.

FIG. 10 shows results of measurements of LV Diastolic Volume pre and post myoblast transplantation in the CABG and Cell Transplantation Group of patients.

FIG. 11 shows results of measurements of LV Dimension pre and post myoblast transplantation in the CABG and Cell Transplantation Group of patients.

### DEFINITIONS

Throughout the specification, several terms are employed that are defined in the following paragraphs.

The term “subject” and “individual” are used herein interchangeably. They refer to a human or another mammal (e.g., a rabbit, monkey, dog, cat, sheep, pig, and the like) that suffers from heart tissue deficiency, damage and/or loss. The deficiency, damage and/or loss may be natural (e.g., resulting from a disease, or congenital defect) or, alternatively, the deficiency, damage and/or loss may be induced (for example in the case of an animal study). In certain preferred embodiments, the subject is a human.

The terms “cardiac damage”, “cardiac dysfunction”, and “condition characterized by insufficient cardiac function or cardiac dysfunction” are used herein interchangeably. They include any impairment or absence of a normal cardiac function or presence of an abnormal cardiac function. Abnormal cardiac function can be the result of a congenital defect, a disease, an injury, and/or the aging process. As used herein, abnormal cardiac function includes morphological and/or functional abnormality of a cardiomyocyte or a population of cardiomyocytes. Non-limiting examples of morphological and functional abnormalities include physical deterioration and/or death of cardiomyocytes, abnormal growth patterns of cardiomyocytes, abnormalities in the physical connection between cardiomyocytes, under- or over-production of a substance or substances by cardiomyocytes, failure of cardiomyocytes to produce a substance or substances which they normally produce, and transmission of electrical impulses in abnormal patterns or at abnormal times. Abnormal cardiac function is seen with many disorders including, for example, ischemic heart disease, e.g., angina pectoris, myocardial infarction, chronic ischemic heart disease, hypertensive heart disease, pulmonary heart disease, valvular heart disease, e.g., rheumatic fever, mitral valve prolapse, calcification of mitral annulus, carcinoid heart disease, infective endocarditis, congenital heart disease, myocardial disease, e.g., myocarditis, dilated cardiomyopathy, hypertensive cardiomyopathy, cardiac disorders which result in congestive heart failure, and tumors of the heart, e.g., primary sarcomas and secondary tumors.

As used herein, the term “myocardial ischemia” refers to a lack of oxygen flow to the heart which results in myocardial ischemic damage. As used herein, the term “myocardial ischemic damage” includes damage caused by reduced blood flow to the myocardium. Examples of causes of myocardial ischemia and myocardial ischemic damage include, but are not limited to, decreased aortic diastolic pressure, increased intraventricular pressure and myocardial contraction, coronary artery stenosis (e.g., coronary ligation, fixed coronary stenosis, acute plaque change (e.g., rupture, hemorrhage), coronary artery thrombosis, vasoconstriction), aortic valve stenosis and regurgitation, and increased right atrial pressure. Non-limiting examples of adverse effects of myocardial ischemia and myocardial ischemic damage include: myocyte damage (e.g., myocyte cell loss, myocyte hypertrophy, myocyte cellular hyperplasia), angina (e.g., stable angina, variant angina, unstable angina, sudden cardiac death), myocardial infarction, and congestive heart failure. Damage due to myocardial ischemia may be acute or chronic, and consequences may include scar formation, cardiac remodeling, cardiac hypertrophy, wall thinning, and associated functional changes. The existence and etiology of acute or chronic myocardial damage and/or myocardial ischemia may be detected or diagnosed using any of a variety of methods and techniques well known in the art including, e.g., non-invasive imaging, angiography, stress testing, assays for cardiac-specific proteins such as cardiac troponin, and clinical symptoms. These methods and techniques as well as other appropriate techniques may be used to determine which subjects are suitable candidates for the treatment methods of the present invention.

The term “treating”, as used herein, includes reducing or alleviating at least one adverse effect or symptom of myocardial damage or dysfunction. In particular, the term applies to treatment of a disorder characterized by myocardial ischemia, myocardial ischemic damage, cardiac damage, or insufficient cardiac function. Adverse effects or symptoms of cardiac disorders are numerous and well-characterized. Examples of adverse effects or symptoms include, but are not limited to, dyspnea, chest pain, palpitations, dizziness, syncope, edema, cyanosis, pallor, fatigue, and death. For additional examples of adverse effects of symptoms of a wide variety of cardiac disorders, see, for example, S. L. Robbins et al., in: “Pathological Basis of Disease”, 1984, W.B. Saunders Co: Philadelphia, Pa., pp. 547-609; and S. A. Schroeder et al., in: “Current Medical Diagnosis and Treatment”, 1992, Appleton & Lange: Norwalk: CT, pp. 257-356.

The terms “delivering”, “administering”, “introducing”, “transplanting”, and “injecting” are used herein interchangeably. They refer to the placement of a cell composition according to the method of the invention into a subject's heart using a catheter-based delivery system which results in localization of the cells of the composition at a desired site (e.g., the site of cardiac damage in the subject).

The terms “skeletal myoblast” and “skeletal myoblast cell” are used herein interchangeably and refer to a precursor of myotubes and skeletal muscle fibers. The term “skeletal myoblasts” also includes satellite cells, mononucleate cells in close contact with muscle fibers in skeletal muscle. Satellite cells lie near the basal lamina of skeletal muscle myofibers and can differentiate into myofibers. As discussed herein, preferred cell compositions for use in the inventive methods comprise skeletal myoblasts and lack detectable myotubes and muscle fibers.

The term “cardiomyocyte” includes a muscle cell which is derived from cardiac muscle. Such cells have one nucleus and are, when present in the heart, joined by intercalated disc structures.

The term “cell proliferation” refers to an expansion of a population of cells by the division of single cells into two daughter cells. The term “cell differentiation”, as used herein, refers to the elaboration of particular characteristics that are expressed by an end-stage cell type or a cell en route to becoming an end-stage cell (i.e., a specialized cell). The term “directed differentiation” refers to a process of manipulating cell culture conditions to induce differentiation into a particular cell type. The term “cell trans-differentiation” refers to the process by which a cell changes from one state of differentiation to another.

The term “stem cell” refers to a relatively undifferentiated cell that has the capacity for sustained self-renewal, often throughout the lifetime of a human or other mammal, and the potential to give rise to differentiated progeny (i.e., to different types of specialized cells). An “embryonic stem cell” is a stem cell derived from a group of cells called the inner cell mass, which is part of the early (4 to 5 days old) embryo called the blastocyst. Once removed from the blastocyst, the cells of the inner cell mass can be cultured into embryonic stem cells. In the laboratory, embryonic stem cells can proliferate indefinitely, a property that is not shared by adult stem cells. An “adult stem cell” is an undifferentiated cell found in a differentiated (specialized) tissue. Adult stem cells are capable of making copies of themselves for the lifetime of the organism. Adult stem cells usually divide to generate progenitor or precursor cells, which then differentiate or develop into “mature” cell types that have characteristic shapes and specialized functions. Sources of adult stem cells include bone marrow, blood, the cornea and retina of the eye, brain, skeletal muscle, dental-pulp, liver, skin, the lining of the gastrointestinal tract, and pancreas. As used herein, the term “plasticity” refers to the ability of an adult stem cell from one tissue to generate the specialized cell type(s) of another tissue.

As used herein, the term “isolated” refers to a cell which has been separated from at least some components of its natural environment. This term includes gross physical separation of the cell from its natural environment (e.g., removal from the donor). Preferably, “isolated” includes alteration of the cell's relationship with the neighboring cells with which it is in direct contact by, for example, dissociation. The term “isolated” does not refer to a cell which is in a tissue section, is cultured as part of a tissue section, or is transplanted in the form of a tissue section. When used to refer to a population of muscle cells, the term “isolated” includes populations of cells which result from proliferation of the isolated cells of the invention.

A cell is “derived from” a subject or sample if the cell is obtained from the subject or sample or if the cell is the progeny or descendant of a cell that was obtained from the subject or sample. A cell that is derived from a cell line is a member of that cell line or is the progeny or descendant of a cell that is a member of that cell line. A cell derived from an organ, tissue, individual, cell line, etc, may be modified in vitro after it is obtained. Such a modified cell is still considered to be derived from the original source.

The terms “approximately” or “about”, as used herein in reference to a number are taken to include numbers that fall within a range of 2.5% in either direction of (i.e., greater than or less than) the number.

As used herein, the term “essentially free of” indicates that the relevant missing item (e.g., cell) is undetectable using either a detection procedure described herein or a comparable procedure known to one of ordinary skill in the art.

As used herein, the term “engraft” includes the incorporation of transplanted muscle cells or muscle cell compositions of the invention into heart tissue with or without the direct attachment of the transplanted cell to a cell in the recipient heart (e.g., by the formation of desmosones or gap junctions).

As used herein, the term “angiogenesis” includes the formation of new capillary vessels in heart tissue, for example, into which cells are transplanted according to the present invention. Cell compositions used in the invention, when transplanted into an ischemic area, preferably enhance angiogenesis. Angiogenesis can occur, for example, as a result of the act of transplanting the cells, as a result of the secretion of angiogenic factors from the cells, and/or as a result of the secretion of endogenous angiogenic factors from the heart tissue.

### DETAILED DESCRIPTION OF CERTAIN PREFERRED EMBODIMENTS

As mentioned above, the present invention provides systems and methods for the minimally invasive treatment of heart tissue damage, deficiency and/or loss, especially in patients suffering from disorders characterized by insufficient cardiac function or cardiac dysfunction.

### I—Cell Composition

The methods of the invention include the delivery of a cell composition to the myocardium of a subject suffering from cardiac dysfunction. Cells that can be transplanted using the inventive therapeutic methods include skeletal myoblasts and/or cardiomyocytes. Cells can be derived from any suitable mammalian source (e.g., human, rabbit, monkey, dog, pig, sheep, and the like) and from a donor of any gestational age (e.g., they can be adult cells, adult stem cells, neonatal cells, fetal cells, or embryonic stem cells).

The cells used in the inventive transplantations may be derived from a single individual, from different individuals of the same species, or from individuals of different species. However, in certain preferred embodiments, the cells are human cells and are used for transplantation into the same individual from which they were derived or for transplantation into an allogeneic subject. Ideally, a biopsy of the patient's own tissue is obtained. Cells can be isolated from a healthy tissue adjacent defective tissue, or from other sites of healthy tissue in the patient. Cells may be isolated by any suitable method. For example, cardiomyocytes may be harvested from a healthy region of the heart of a patient undergoing an open chest procedure, such as coronary artery bypass grafting (CABG) or left ventricular assist device (LVAD) implantation, and used for future transplantation(s) into the damaged/defective area(s) of the patient's myocardium. Alternatively or additionally, skeletal muscle cells may be isolated from the patient's limb muscle, such as biceps and quadriceps, to prepare skeletal myoblasts. One major advantage of autologous cells is that they do not elicit an immunologic reaction in the recipient. Therefore, autologous transplantation is often preferred, particularly when the patient's cells are genetically normal with respect to muscle functioning, and the patient's myocardium is not strongly damaged. In other embodiments, cells of the same species and preferably of the same immunological profile can be obtained, for example, from a patient's close relative or another donor. In this case, tissue rejection is alleviated by using a schedule of steroids and other immunosuppressant drugs such as cyclosporine.

Cellular compositions used in the inventive transplantations may be varied depending on the cardiac dysfunction to be treated, the severity of the dysfunction, and/or the nature of previous cell injection(s) or transplantation(s) received by the patient. In certain embodiments, the cells of the composition are essentially of a single cell type. In other embodiments, the cells of the composition are of at least two different cell types. For example, a cell composition may consist essentially of skeletal myoblasts or of cardiomyocytes. Alternatively, a cell composition may comprise skeletal myoblasts, cardiomyocytes, fibroblasts, and/or stem cells.

While not wishing to be bound by any particular theory, it is possible that the presence of fibroblasts, cardiomyocytes and/or stem cells in the cell composition may enhance myoblast survival, proliferation, differentiation, functionality, integration, or longevity into the host tissue, and/or may increase engraftment efficiency, enhance graft strength, and/or favor new blood vessel formation, etc. Thus, it may be desirable to include varying percentages of these cells within the cell compositions.

In certain embodiments, the cell composition to be used for transplantation according to the present invention comprises at least about 20%, 25%, 30%, 35%, 40%, 45%, 50%, 55%, 60%, 65%, 70%, 75%, 80%, 85%, 90%, 95%, 96%, 97%, 98% or 99% myoblasts. Compositions having these percentages of myoblasts can be made (e.g., using standard cell sorting techniques to obtain purified populations of cells). The purified populations of myoblasts can then be mixed to obtain the desired percentage of myoblasts. Alternatively, cell compositions comprising the desired percentage of myoblasts can be obtained by culturing a freshly isolated population of skeletal myoblasts in vitro for a limited number of population doublings such that the percentage of myoblasts in the composition falls within the desired range.

In other embodiments, the cell composition to be used for transplantation according to the present invention comprises skeletal myoblasts and fibroblasts. Preferably, the cell composition comprises from about 20% to about 70% myoblasts, for example, from about 40-60% myoblasts or about 50% myoblasts. Myoblast culture is generally associated with fibroblast contamination. Therefore, fibroblasts present in a cell composition may be produced by preparing myoblasts. Alternatively, myoblasts may be combined with fibroblasts derived from a tissue source other than muscle tissue (for example, with fibroblasts derived from skin).

In yet other embodiments, the cell composition to be used for transplantation according to the present invention comprises skeletal myoblasts and cardiomyocytes. Preferably, the composition comprises from about 20% to about 70% myoblasts, for example, from about 40-60% myoblasts or about 50% myoblasts. After in vitro expansion, myoblasts and cardiomyocytes may be combined to obtain the desired composition.

In still other embodiments, the cell composition to be used for transplantation according to the present invention comprises skeletal myoblasts and stem cells. Preferably, the composition comprises from about 20% to about 70% myoblasts, for example, from about 40-60% myoblasts or about 50% myoblasts. After preparation, myoblasts and stem cells may be combined to obtain the desired composition.

The relative percentage of myoblasts and other cells in a cell composition can be determined, for example, by staining one or both populations of cells with a cell specific marker and determining the percentage of cells in the composition which express the marker (e.g., using standard techniques such as FACS analysis).

Preferably, a cell composition to be used according to the present invention comprises muscle cells that have been cultured in vitro for less than a certain number of population doublings prior to transplantation. For example, human muscle cells may be permitted to undergo less than about 20, less than about 15, less than about 10, less than about 5, or between about 1 and about 5 population doublings prior to transplantation. The optimal number of doublings may vary depending upon the mammal species from which the cells were isolated. Determination of the optimal number of doublings is easily performed by one skilled in the art.

Cells and cell compositions to be used in the therapeutic methods of the present invention can be used fresh, or can be cultured and/or cryopreserved prior to their use in transplantation.

### Skeletal Myoblasts

The skeletal myoblasts to be used in the therapeutic methods of the present invention may be prepared using any suitable procedure. Different techniques of isolation, expansion and purification have been reported (see, for example, U.S. Pat. Nos. 5,833,978; 5,538,722; 5,466,676; and 6,337,184, each of which is incorporated herein by reference in its entirety). A preferred method of preparation, developed by the Applicants, is disclosed in U.S. Pat. No. 6,673,604 and U.S. Appln. No. 2003/0113301, which are both incorporated herein by reference in their entirety.

In most methods, a muscle sample (or other sample) that contains muscle progenitor cells such as satellite cells is obtained from a donor (the future recipient of the cell composition or another individual). Biopsies of 0.5 to 6 grams are generally obtained. The tissue may be immediately treated/processed or it may be cryopreserved for future use. If desired, the site from which the muscle tissue is obtained may be stimulated prior to tissue harvest in order to increase the final number of myoblasts. Such stimulation may be mechanical and/or by treatment with compounds such as growth factors.

Harvested tissue can be placed into a digestion medium (e.g., containing one or more proteases such as collagenase, elastase, endoproteinase, trypsin, and the like, and optionally EDTA) and cut into pieces (e.g., using a surgical blade). The biopsy pieces can be teased into fine fragments (e.g., using the needle tips of two tuberculin syringe needle assemblies), and connective tissue may be removed (e.g., using visual inspection). If desired, such connective tissue may be cultured separately in order to obtain fibroblasts. Cells released into the digestion medium may be collected (e.g., by vortexing). Several digestion steps may be performed using different proteases, different concentrations of proteases, different digestion times, and/or different digestion temperatures, in order to increase the number of cells released by the harvested tissue. The absolute and relative yield of myoblasts, fibroblasts, etc, at each step may be estimated (e.g., by visual inspection). Alternatively or additionally, the cells released may be sorted (e.g., using fluorescence activated cell sorting), for example to select populations of cells exhibiting desired percentages of myoblasts and/or fibroblasts.

Isolated cells are then expanded in vitro prior to transplantation using standard cell culture techniques and conditions. In general, the cells are grown in culture in a medium suitable to support the growth of the cells. Media which can be used to support growth and/or viability of muscle cells are known in the art and include mammalian cell culture media, such as those available, for example, from Gibco/BRL (Invitrogen, Gaithersburg, Md.). The medium can be serum-free but is preferably supplemented with animal serum such as fetal calf serum. Optionally, growth factors can be included. Media which are used to promote proliferation of muscle cells and media which are used for maintenance of cells prior to transplantation can differ. In some embodiments, a preferred growth medium for muscle cells is MCDB 120 comprising dexamethasone (e.g., 0.39 μg/mL), Epidermal Growth Factor (EGF) (e.g., 10 ng/mL), and fetal calf serum (e.g., 15%); and a preferred medium for muscle cell maintenance is DMEM supplemented with protein (e.g., 10% horse serum). Other exemplary media are taught, for example, in R. R. Henry et al., Diabetes, 1995, 44: 936-946; and WO 98/54301 (each of which is incorporated herein by reference in its entirety).

Skeletal myoblasts may be seeded on laminin coated plates for expansion in myoblast growth Basal Medium containing 10% FBS, dexamethasone and EGF. Alternatively, skeletal myoblasts may be seeded on collagen coated plates for expansion in myoblast growth Basal Medium containing 10% FBS, dexamethasone and FGF. Alternatively, skeletal myoblasts may be seeded on the surface of a plate without any coating and grown in myoblast growth Basal Medium containing 10% FBS, dexamethasone and FGF. The surface can be a petri dish or a surface suitable for large scale culture of cells. The culture time in vitro is generally less than a maximum of about 14 days and is preferably about 7 days. After expansion, myoblasts are harvested using 0.05% trypsin-EDTA and washed in medium containing FBS. Where the percentage of myoblasts in the harvested cell population differs from that desired for the transplantable cell composition, the percentages may be adjusted by cell sorting and/or by combining different cell populations.

The isolated cells may be expanded in culture under conditions selected to minimize or reduce the likelihood of myoblast fusion. For example, it may be desirable to maintain the cells in a subconfluent state (e.g., less than approximately 50% confluence, less than approximately 50% to 75% confluence, or less than approximately 75% to 90% confluence). To ensure that cells do not exceed desired confluence, they may be passaged at appropriate time intervals.

### Cardiomyocytes

Cardiomyocytes may be prepared by any suitable method. Methods have been reported for the isolation and expansion of cardiomyocytes from different mammal species including, for example, human (P. P. Nanasi et al., Cardioscience, 1993, 4: 111-116; S. D. Bird et al., Cardiovasc. Res., 2003, 58: 423-434; E. Messina et al., Circ. Res., 2004, 95: 911-921); dog (J. C. Hisch et al., Methods Mol. Biol., 2003, 219: 145-157); and rat (R. K. Li et al., Ann. Thorac. Surg., 1996, 62: 654-661).

Isolated cardiomyocytes are grown in vitro in culture using standard cell culture techniques and conditions. Media for the culture of mammalian cardiac cells are known in the art (see, for example, S, N. Mohamed et al., In Vitro Cell and Develop. Biol., 1983. 19: 471-478; P. Libby, J. Mol. Cell. Cardiol., 1984. 16: 803-811; D. L. Freerksen et al., J. Cell. Physiol., 1984, 120: 126-134; G. Kessler-Icekson et al., Exp. Cell Res., 1984. 155: 113-120; J. S. Karliner et al., Biochem. Biophys. Res. Comm., 1985. 128: 376-382; T. Suzuki et al., FEBS Letters, 1990, 268: 149-151; T. Suzuki et al., J. Cardiov. Pharmacol., 1991, 17: S182-S186; T. Suzuki et al., J. Mol. Cell. Cardiol., 1997, 29: 2087-2093, each of which is incorporated herein by reference in its entirety). Different factors and agents may be added to the medium.

When cardiomyocytes are grown in culture, at least about 20%, preferably at least about 30%, more preferably at least about 40%, yet more preferably about 50%, and most preferably at least about 60% or more of the cardiomyocytes express cardiac troponin and/or myosin, among other cardiac-specific cell products.

### Stem Cells

In certain embodiments, the cell composition to be delivered to the heart of a subject suffering from cardiac dysfunction comprises stem cells. Stem cells are known to provide a virtually never-ending supply of cells for tissue engineering and clinical applications. The advantage of embryonic stem cells as a cell source, include virtually indefinite growth and differentiation potential that encompasses all cells and tissues. Specific differentiation in vitro into cells with the phenotypes of cardiomyocytes, neural cells, and insulin producing beta cells has been demonstrated. Muscle cells have also been derived from embryonic stem cells (M. G. Klug et al., J. Clin. Invest., 1996, 98: 216-224; J. Dinsmore et al., Cell Transplantation, 1996, 5: 131-143).

The discovery that some stem cell populations isolated from adult tissues exhibit some degree of plasticity has opened new avenues for basic biological research and the development of new therapies and clinical tools. The so-called adult stem cells can be derived from a variety of specific tissues to provide, for example, mesenchymal, neuronal, and endothelial cells.

There has been a plethora of reports suggesting that primitive stem cells within whole bone marrow possess greater functional plasticity than was previously suspected. After bone marrow transplantation into animal models, donor-derived stem cells have been found in such diverse non-hematopoietic tissues as skeletal muscle (G. Ferrari et al., Science, 1998, 279: 1528-1530), cardiac muscle (R. E. Bittner et al., Anat. Embryol. 1999, 199: 391-396), liver (B. E. Petersen et al., Science, 1999, 284: 1168-1170), vascular endothelium (T. Asahara et al., Science, 1997, 275: 964-967) and brain (E. Mezey et al., Science, 2000, 290: 1779-1782; T. R. Brazelton et al., Science, 2000, 290: 1775-1779). Similarly, enriched or purified hematopoietic stem cells have been reported to generate skeletal muscle (E. Gussoni et al., Nature, 1999, 401: 390-394), cardiac muscle (K. A. Jackson et al., J. Clin. Invest. 2001, 107: 1395-1402; D. Orlic et al., Proc. Natl. Acad. Sci. USA, 2001, 98: 10344-10349; D. Orlic et al., Science, 2001, 410: 701-705), endothelial cells (K. A. Jackson et al., J. Clin. Invest. 2001, 107: 1395-1402), liver hepatocytes and bile duct (E. Lagasse et al., Nat. Med. 2000, 6: 1229-1234), as well as multiple epithelial tissues (D. S. Krause et al., Cell, 2001, 105: 369-377).

Stem cells derived from bone marrow, whether multipotent hematopoietic stem cells or other tissue specific stem cells resident in the bone marrow, have a major advantage over stem cells from other organ in that they are well defined and easy to isolate. Moreover, transplantation of bone marrow hematopoietic stem cells has been found to induce donor tolerance, allowing trans-differentiation or transplantation of other tissue specific stem cells from the same donor without the need from prolonged immunosuppression of the recipient.

In particular, mesenchymal stem cells, which reside within the bone marrow cavity, have been shown, both in culture and following injection into particular tissues in mammals, to give rise to a range of cell types including cardiac and skeletal muscle cells (K. W. Liechty et al., Nat. Med. 2000, 6: 1282-1286; M. F. Pittenger et al., Science, 1999, 284: 143-147). Isolation, purification, and culture expression of human mesenchymal stem cells have been described, for example, in U.S. Pat. No. 6,387,369 (which is incorporated herein by reference in its entirety).

The microenvironment (including contact with surrounding cells, formation of extracellular matrix, nature of local milieu as well as presence of growth and differentiation factors) plays a role in determining the stem cells' function. Stem cells can be used as such in the cell compositions to be transplanted. Alternatively, stem cell cultures can be treated under conditions and/or in the presence of specific factors and agents that drive differentiation along a predetermined lineage. A selectable marker under the control of a lineage-specific promoter, for example, a transcription factor that is switched on early during lineage-specific differentiation, may be inserted into the stein cells. The selectable marker will then be expressed in cells undergoing differentiation into the lineage in question, and, by applying the selective agent, it is possible to kill off other cell types in the cultures.

For example, U.S. Pat. No. 6,387,369 describes a series of specific treatments applicable to mesenchymal stem cells to induce expression of cardiac specific genes. The conditions that are disclosed are effective on rat, canine, and human mesenchymal stem cells. Mesenchymal stem cells that progress towards cardiomyocytes, first express proteins found in fetal cardiac tissue and then proceed to adult forms. Detection of expression of cardiomyocyte-specific proteins can be achieved by using antibodies to, for example, myosin heavy chain monoclonal antibody MF-20 or sarcoplasmic reticulum calcium ATPase.

### Modifications of Cells

Before transplantation into the heart of a subject suffering from cardiac dysfunction, cells may be modified. For example, antigens on the surface of a cell may be altered in such a way that upon transplantation, lysis of the cell is inhibited. Alteration of an antigen can induce immunological non-responsiveness or tolerance, thereby preventing the inducing of the effector phases of an immune response (e.g., cytotoxic T cell generation, antibody production, etc.) which are ultimately responsible for rejection of foreign (i.e., allogeneic or xenogeneic) cells in a normal immune response. Antigens that can be altered to achieve this goal include, for example, MHC class I antigens, MHC class II antigens, LFA-3 and ICAM-1. Preferred methods for altering an antigen on a donor cell to inhibit an immune response against the cell have been disclosed in U.S. Pat. No. 6,673,604 and U.S. Pat. Application No. 2003/0113301 (which are incorporated herein by reference in their entirety).

Alternatively or additionally, cells to be transplanted in a patient's damaged/defective myocardium according to the present invention can be genetically modified before transplantation. For example, the cells may be modified to express a gene product (i.e., cells may be treated in a manner that results in the production of a gene product by the cell). Preferably, the cell does not express the gene product prior to modification. Alternatively, modification of the cell may result in an increased production of a gene product already expressed by the cell or may result in production of a gene product (e.g., an antisense RNA molecule) which decreases production of another, undesirable gene product normally expressed by the cell.

For example, cells may be genetically modified to more closely resemble cardiac muscle cells in phenotype. Such “cardiac-like cells” can be characterized, for example, by a change in their physiology (e.g., they may have a slower twitch phenotype, a slower shortening velocity, use of oxidative phosphorylation for ATP production, expression of cardiac forms of contractile proteins, higher mitochondrial content, higher myoglobin content, and/or greater resistance to fatigue than skeletal muscle cells), and/or the production of molecules which are normally not produced by skeletal muscle cells or which are normally produced in low amounts by skeletal muscle cells (e.g., those proteins produced from genes encoding the myocardial contractile apparatus and the Ca²⁺ ATPase associated with cardiac slow twitch, phospholamban, and/or β-myosin heavy molecules).

Alternatively or additionally, cells may be genetically modified to express a gene product to be supplied to the subject receiving the transplantation. Examples of gene products that can be delivered to a subject via a genetically modified muscle cells include gene products that can prevent future cardiac disorders, such as growth factors which encourage blood vessels to invade the heart muscle (e.g., Vascular Endothelial Growth Factor (VEGF), Fibroblast Growth Factor (FGF) 1, FGF-2, Transforming Growth Factor beta (TGF-β), and angiotensin). Other gene products that can be delivered to a subject via a genetically modified cardiomyocyte include factors which promote cardiomyocyte survival, such as FGF, TGF-β, IL-10 (Interleukin 10), CTLA 4-Ig (cytotoxic T lymphocyte-associated antigen 4 immunoglobulin), and bcl-2. (B-cell leukemia/lymphoma 2)

Mesenchymal stem cells may also be genetically modified or engineered to express proteins of importance for the differentiation and/or maintenance of striated skeletal muscle cells. Exemplary proteins include growth factors (e.g., TGF-β, Insulin-Like Growth Factor 1 (IGF-1), FGF), myogenic factors (e.g., myoD, myogenin, myogenic factor 5 (Myf5), Myogenic Regulatory Factor (MRF)), transcription factors (e.g., GATA-4), cytokines (e.g., cardiotropin-1), members of the neuregulin family (e.g., neuregulin 1, 2, and 3) and homeobox genes (e.g., Csx, tinman, NKx family).

Cells to be transplanted may, additionally or alternatively, be engineered to recombinantly express an angiogenic gene product, such as, VEGF (M. Asano et al., Jpn. J. Cancer Res., 1999, 90: 93-100), IGR-I, IGF-II, TGF-β1, platelet-derived growth factor-β (PDGF-β), or an agent that acts indirectly to induce an angiogenic agent, such as, for example, fibroblast growth factor-4 (FGF-4) (C. F. Deroanne et al., Cancer Res., 1997, 57: 5590-5597).

### Cell Characterization

Cell viability can be determined using standard techniques including histology, quantitative assessment with radioisotopes, or visual observation using a light or scanning electron microscope or fluorescent microscope. The biological function of the cells can be determined using a combination of the above techniques and/or standard functional assays.

### II—Catheter Delivery

In the therapeutic methods of the present invention, the skeletal myoblasts, optionally combined with fibroblasts, cardiomyocytes and/or stem cells, as described above, are transplanted into a subject's myocardium at or near a site of tissue deficiency, damage and/or loss, using a catheter-based delivery system inserted into the patient's venous system. In certain preferred embodiments, the recipient subject will have been diagnosed to have region(s) of damaged/defective cardiac tissue such as ischemic tissues, fibrotic tissues or scar tissues. The use of a catheter for cell transplantation into a patient's myocardium according to the present invention precludes more invasive methods of delivery, which would require opening of the chest cavity.

### Identification of Damaged/Defective Cardiac Tissue

In certain embodiments, the inventive methods include a step of identifying area(s) of a subject's heart in need of treatment. Identification of damaged and/or defective cardiac tissue can be performed by any suitable method. Multiple technologies and approaches are available today for the clinician to assess normal, ischemic non-viable, and ischemic-viable myocardial tissue. These include, but are not limited to, localized blood flow determinations, local electrical and mechanical activity, nuclear and imaging cardiology (e.g., MRI, SPECT or PET), echocardiography stress test, coronary angiography, and ventriculography. Any one of these techniques or any combination thereof may be used in the practice of the present invention to identify and target specific area(s) of the heart that exhibit(s) tissue damage, deficiency and/or loss.

For example, identification of damaged/deficient region(s) of a subject's heart may be carried out by a technique called “mapping of the heart”. The theory behind cardiac mapping is that certain types of cardiac disorders caused by areas of abnormal heart tissue, interrupt the heart's normal electrical systems. Cardiac mapping was reported as early as 1915 (T. Lewis and M. A. Rothschild, Philos. Trans. R. Soc. London B: Biol. Sci., 1915, 206: 181-226) and implies the registration of the electrical activation sequence by recording extracellular electrograms. More recent techniques (see, for example, U.S. Pat. No. 6,447,504, which is incorporated herein by reference in its entirety) provide simultaneous electrophysiological and spatial information. In these techniques, the data is acquired using one or more catheters that are advanced into the heart. These catheters usually have electrical and location sensors in their distal tips. Some of the catheters have multiple electrodes on a three-dimensional structure and others have multiple electrodes distributed over a surface area. One example of the later catheter may be a sensor electrode distributed on a series of circumferences of the distal end portion, lying in planes spaced from each other. In addition to using electrical potentials in the heart tissue to characterize the heart's condition, these techniques can also use electromechanical mapping and/or ultrasonic mapping to localize the viable and the non-viable regions of the heart. Furthermore, when ultrasonic mapping is used, the ultrasound waves may help determine the thickness of the heart tissue in the vicinity of the probe.

One of the preferred suitable cardiac mapping systems to be used in the present invention is the NAVI-STAR® diagnostic/ablation deflectable tip catheter equipped with the CARTO™ EP Navigation System (provided by BioSense Webster, Inc., Diamond Bar, Calif.), which is a non-fluoroscopic cardiac mapping system that enables the generation of 3-D electroanatomical maps of the heart chambers. More specifically, CARTO™ is a catheter-based system that is generally introduced using an 8F or 9F femoral sheath and placed in the patient's left ventricle. CARTO™ is comprised of miniature passive-magnetic field sensors, an external ultra-low magnetic field emitter, (or location pad), and a processing unit. The miniature magnetic field sensors are located at the tips of a mapping/ablation catheter (NAVI-STAR®) and a reference catheter (which may be taped securely to the patient's back). Three magnetic field emitters are situated under the catheterization table and emit three different frequencies. The sensors receive the emitted low-intensity magnetic fields and transmit them along the catheter shaft to the main processing unit. The processing unit collects and analyzes data on the amplitude, frequency, and phase of the magnetic fields to determine the precise location of the mapping/ablation catheter tip (x, y and z) and its orientation (roll, pitch and yaw) within the fields. The three-dimensional geometry of the cardiac chamber is generated, and the system displays the real-time location of the two catheters relative to each other. The electrophysiological information is color-coded and superimposed over the electroanatomical map.

### Catheter-Based Delivery System

Any catheter-based delivery system that allows for the injection of a skeletal myoblast composition into a subject's myocardium at or near the area(s) of cardiac tissue damage or deficiency can be used in the practice of the therapeutic methods of the present invention. In certain embodiments, the catheter is introduced percutaneously (e.g., into the femoral artery or another blood vessel) and routed through the vascular system to the subject's myocardium where it is used to deliver the cell composition via a needle that is extruded from the end of the catheter. In other embodiments, the catheter reaches the heart through minimal surgical incision (e.g., limited thoracotomy, which involves an incision between the ribs).

Several catheters have been designed in order to precisely deliver agents to a damaged region within the heart, for example, an infarct region (see, for example, U.S. Pat. Nos. 6,102,926; 6,120,520; 6,251,104; 6,309,370; 6,432,119, and 6,485,481, each of which is incorporated herein by reference in its entirety). The catheter may be guided to the indicated location by being passed down a steerable or guidable catheter having an accommodating lumen (see, for example, U.S. Pat. No. 5,030,204) or by means of a fixed configuration guide catheter (see, for example, U.S. Pat. No. 5,104,393) Alternatively, the catheter may be advanced to the desired location within the heart by means of a deflectable stylet (see, for example WO 93/04724), or a deflectable guide wire (see, for example, U.S. Pat. No. 5,060,660).

Preferably, the catheter is coupled to a cardiac mapping system, which allows determination of the location and extent of the damaged/defective zone(s) (as described above). Once an area in need of treatment is identified, the steering guide may be pulled out leaving the needle at the site of injection. Part or all of the cell composition is then sent down the lumen of the catheter and injected into the myocardium. The catheter is retracted from the patient when all the injections have been performed.

The needle element may be ordinarily retracted within a sheath at the time of guiding the catheter into the patient's heart to avoid damage to the venous system and/or the myocardium. At the time of injection, the needle is extruded from the tip of the catheter. During injection, the needle protrudes less than 10 mm, less than 7.5 mm or less than 5 mm into an adult heart muscle wall. Depending on the site of injection, the maximum length may be altered. For infants and children, the protrusion depth is correspondingly less, as determined by the actual or estimated wall thickness. The needle gauge used in transplantation of the cells can be, for example, 25 to 30.

In preferred embodiments, the catheter used to deliver the cell composition to the myocardium is configured to include a feedback sensor for mapping the penetration depth and location of the needle insertion. The use of a feedback sensor provides the advantage of accurately targeting the injection location. Depending on the type and severity of the cardiac tissue damage, the target location for delivering the cell composition may vary. For example, an optimal treatment may require multiple small injections within a damaged/defective region where no two injections penetrate the same site. Alternatively, the target location may remain the same of successive cell transplantation procedures.

A suitable catheter that may be used in the present invention is the NOGA™ Injection Catheter system (Biosense Webster, Inc.). This catheter is a multi-electrode, percutaneous catheter with a deflectable tip and injection needle designed to inject agents into the myocardium. The tip of the Injection Catheter is equipped with a Biosense location sensor and a retractable, hollow 27-gauge needle for fluid delivery. The injection site is indicated in real-time on the heart map, allowing for precise distribution of the injections. Local electrical signals are obtained to minimize catheter-tip trauma.

### III—Uses and Applications of the Inventive Methods

The present invention provides methods for the minimally invasive treatment of cardiac tissue damage, deficiency and/or loss, especially in patients suffering from disorders characterized by insufficient cardiac function or cardiac dysfunction. In certain embodiments, a cell composition comprising autologous skeletal myoblasts and, optionally, fibroblasts, cardiomyocytes and/or stem cells, is delivered to a subject's myocardium at or near the site of tissue damage, deficiency or loss, using an intravascular catheter with a deployable needle. Preferably, the cell transplantation is performed after identifying a region of the subject's myocardium in need of treatment (for example, cardiac tissue damage by ischemia, fibrotic tissue or scar tissue).

### Medical Indications

Medical indications for the inventive therapeutic methods include, but are not limited to, coronary heart disease, cardiomyopathy, endocarditis, congenital cardiovascular defects, congestive heart failure and myocardial infarction. A final common pathway of many cardiovascular diseases is irreversible damage of the cardiac muscle tissue. As already mentioned above, this effect is generally attributed to the inability (or weak capacity) of cardiac cells to replicate after injury (M. H. Soonpaa and L. D. Field, Circ. Res., 1998, 83: 15-26) as well as to the lack of substantial source of resident stem cells in the myocardium.

Excessive loss of cardiomyocytes due to ischemia (deficiency of blood flow) and formation of scar tissue are, for example, observed after myocardial infarction. Infarcts occur when a coronary artery becomes obstructed and no longer supplies blood to the myocardial tissue. The damage of myocardial infarction is generally progressive (D. L. Mann, Circulation, 1999, 100: 999-1008). However, the consequences are often severe and disabling. Immediate hemodynamic effects are followed by three major processes: infarct expansion, infarct extension, and ventricular remodeling. The magnitude and clinical significance of these processes highly depend on the size and location of the myocardial infarction (H. F. Weisman and B. Healy, Frog. Cardiovasc. Dis., 1987, 30: 73-110; S. T. Kelley et al., Circulation, 1999, 99: 135-142).

Early after a myocardial infarction, infarct expansion takes place through slippage of the tissue layers, which results in a permanent regional thinning and dilation of the infarct zone. Infarct extension corresponds to additional myocardial necrosis and produces an increase in total mass of infarcted tissue. The presence of infarcted tissue (i.e., scar tissue that is unable to contract during systole) leads to a depression in ventricular function, and eventually to dysfunction in cardiac tissue remote from the site of initial infarction. This greatly exacerbates the nature of the disease and can often progress into advanced stages of congestive heart failure. The third process, ventricular remodeling, usually happens weeks or years after myocardial infarction. It corresponds to a progressive enlargement of the ventricle with depression of ventricular function, and is believed to result from the high stress undergone by tissues surrounding the initial infarction zone (D. K. Bogen et al., Circulation Res., 1980, 47: 728-741; J. Lessick et al., Circulation, 1991, 84: 1072-1086). Deterioration of the ventricular function eventually leads to heart failure (D. L. Mann, Circulation, 1999, 100: 999-1008).

Despite recent advances in the treatment of acute myocardial infarction, the ability to repair extensive myocardial damage and to treat heart failure is limited (D. L. Mann, Circulation, 1999, 100: 999-1008). A possible strategy to restore heart function after myocardial injury is to replace the damaged tissue with healthy tissue. Experiments have shown that the strategy of tissue engineering could be used for regeneration and healing of the infarcted myocardium and for the attenuation of wall stress, infarct expansion and left ventricle dilatation. These beneficial effects could be translated into the prevention of heart failure progression (J. Leor et al., Circulation, 2000, 102: III56-61). However, this strategy requires open chest surgery, a procedure that is performed under general anesthesia and that is generally associated with high risks of complications.

Transplantations of skeletal myoblasts (optionally combined with fibroblasts, cardiomyocytes, and/or stem cells) according to the methods of the present invention may be performed on patients with myocardial infarction, at any stage of the disease (i.e., immediately following diagnosis of the myocardial infarction, as well as before and/or after any of the different phases of the disease, i.e., infarct expansion, infarct extension, and ventricular remodeling).

Other medical indications for the inventive methods of treatment include congenital heart defects. When the heart or blood vessels near the heart do not develop normally before birth, a condition called congenital defect occurs. Most heart defects cause an abnormal blood flow through the heart or obstruct blood flow in the heart and vessels. Congenital heart defects include obstruction defects (such as aortic stenosis, pulmonary stenosis, bicuspic aortic valve, subaortic stenosis, and coarctation of the aorta), septal defects (such as atrial septal defect, Ebstein's anomaly and ventricular septal defect), cyonotic defects (such as tetraology of Fallot, tricuspid atresia and transposition of the great arteries), hypoplastic left heart syndrome and patent ductus arteriosus.

Transplantations of cell compositions according to the present invention may, alternatively, be performed on a patient who has previously undergone a coronary artery bypass graft (CABG) implantation. More than 500,000 coronary artery bypass operations are performed annually in the U.S. alone. Bypass surgery may be needed for various reasons, for example, to restore blood flow to cardiac tissue that has been deprived of blood because of a coronary artery disease, or in the case of an angioplasty that did not sufficiently widen the blood vessel, or because of blockages that cannot be reached by, or are too long or stiff for, angioplasty. In conventional coronary artery bypass graft operation, a piece of vein taken from the leg of the patient, or from an artery from the chest or wrist is attached to the heart artery above and below the narrowed area, thus making a bypass around the blockage. The best results are generally obtained if one of the patient's own vessels is grafted. However, if an autologous vessel cannot be used, a prosthetic vessel may be implanted. These procedures substantially improve symptoms in more than 90% of patients who undergo the treatment. A graft may be placed to any one of the following arteries: left main coronary artery, which supplied the left ventricle of the heart; the left anterior coronary artery, and posterior descending artery.

Transplantation(s) of skeletal myoblasts according to the present invention may be performed at any time following CABG implantation. For example, a cell composition may be injected 2 days, 7 days, 2 weeks, 1 month, 3 months, 6 months or 1 year after CABG implantation. Alternatively or additionally, catheter delivery of a cell composition according to the present invention may be performed while the patient is undergoing CABG implantation (i.e., during the open chest procedure).

Transplantations of cell compositions according to the present invention may, alternatively, be performed on a patient who has previously undergone implantation of a left ventricular assist device (LVAD), also known as “bridge to transplant” or “bridge to recovery” A left ventricular assist device is a battery-operated, mechanical pump-type device which, after being surgically implanted, helps maintain the pumping ability of a deficient heart, thus decreasing the work of the left ventricle. During an open-heart procedure, a surgeon attaches the LVAD to the apex of the left ventricle and to the aorta. When the left ventricle contracts (systole), blood flows into the LVAD pump. When the heart relaxes (diastole), the left ventricle fills with blood, and the blood in the device is pumped into the aorta. The original indication of the LVAD therapy was to allow patients to have an acceptable quality of life while waiting for a donor heart to become available (thence its name of “bridge to transplantation”). However, device removal and long-term therapeutic benefits have been achieved, even in patients with severe chronic heart failure (thence its other name of “bridge to recovery”).

The present Applicants have found that transplanting cells to the heart of a patient undergoing implantation of an LVAD is beneficial to the patient. In particular heart tissue remodeling was observed to improve in the presence of skeletal myoblasts. The present invention provides for the catheter delivery of cell compositions to the myocardium of patients who have previously received a left ventricular assist device. Catheter-based transplantation(s) of skeletal myoblasts according to the presence invention may be performed at any time post-LVAD implantation. For example, a cell composition may be injected 2 days, 7 days, 2 weeks, 1 month, 3 months, 6 months or 1 year after LVAD implantation. Alternatively or additionally, catheter delivery of a cell composition according to the present invention may be performed while the patient is undergoing LVAD implantation (i.e., during the open chest procedure).

Efficacy of the therapeutic methods of the present invention can be monitored by clinically accepted criteria, such as reduction in area(s) occupied by ischemic, fibrotic and/or scar tissue; vascularization of ischemic, fibrotic and/or scar tissue, improvement in developed pressure, systolic pressure, end diastolic pressure, patient mobility and quality of life compared with before transplantation.

Transplantations of skeletal myoblasts (optionally combined with fibroblasts, cardiomyocytes, and/or stem cells) according to the present invention may also be performed in animals, including animals acting as models of human damage or disease that occurs in humans. Heart of small animal models can be cryoinjured by placing a precooled aluminum rod in contact with the surface of the anterior left ventricle wall (C. E. Murry et al., J. Clin. Invest., 1996, 98: 2209-2217; H. Reinecke et al., Circulation, 1999, 100: 193-202; U.S. Pat. No. 6,099,832). In larger animals, cryoinjury can be inflicted by placing a 30-50 mm copper disk probe cooled in liquid nitrogen on the anterior wall of the left ventricle for about 20 minutes (R. C. Chiu et al., Arm. Thorac. Surg., 1995, 60: 12-18). Infarction can be induced by ligation of the left main coronary artery (Q. Li et al., J. Clin. Invest., 1997, 100: 1991-1999). Example 1 and Example 2 describe methods of inducing myocardial infarction in a sheep and swine model, respectively.

A cell composition may be delivered to the site of injury of the animal model's heart using a catheter. Suitability of the treatment may be determined by assessing the degree of cardiac recuperation that follows the transplantation. Cardiac function may be monitored by determining such parameters as left ventricular end-diastolic pressure, developed pressure, rate of pressure rise, and rate of pressure decay. After a certain period of time following transplantation, tissues may be harvested and studied by histology. Cells of the tissue harvested may be tested for their ability to have survived and maintained their phenotype in vivo. The presence and phenotype of the cells can be assessed by immunohistochemistry or ELISA using specific antibody, or by RT-PCR analysis.

### Dosages, Formulations and Administrations.

Skeletal myoblasts, optionally combined with fibroblasts, cardiomyocytes and/or stem cells, are preferably administered suspended in a solution. As used herein, the term “solution” includes a pharmaceutically acceptable carrier or diluent in which the cells are suspended such that they remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions to be used for transplantation can be prepared by incorporating the cells as described above in pharmaceutically acceptable carrier or diluent and, as required, other ingredients (see below), followed by filtered sterilization.

To treat disorders characterized by insufficient cardiac function in a human subject, about 1×10⁶ to about 1×10⁹ cells can be implanted into the heart (e.g., about 1×10⁶ to about 10×10⁶, about 10×10⁶ to about 100×10⁶, about 100×10⁶ to about 500×10⁶, or about 0.5×10⁹ to about 2×10⁹) at each treatment. In cases of repeated dosing, the total number injected cells may exceed 1×10⁹ and reach up to 10×10⁹ in total. Preferably, the composition comprises about 100×10⁶ cells/mL (e.g., about 20×10⁶ to about 300×10⁶ cells/mL, preferably about 30×10⁶ to about 250×10⁶ cells/mL, more preferably about 50×10⁶ to about 200×10⁶ cells/mL). The cells may be injected into the myocardium in separated injections of about 0.05 mL to about 1.5 mL, preferably about 0.1 mL to about 1 mL, and more preferably from 0.1 mL to 0.5 mL injection volumes of cell composition. Between 2 to 100, or between 4 to 50, or between 10 and 35 injections can be made for a given heart treatment.

Patients may undergo one or more treatments according to the present invention. The cellular composition of the cell suspension administered may vary from patient to patient, and/or from treatment to treatment for a patient receiving multiple transplantations over time.

It is generally preferred that at least about 5%, preferably at least about 10%, more preferably at least about 20%, yet more preferably at least about 30%, still more preferably at least about 40%, and most preferably at least about 50% or more of the cells remain viable after administration into a subject. The period of viability of the cells after administration to a subject can be as short as a few hours (e.g., 24 hours, to a few days, to as long as a few weeks to years).

The cell composition can comprise, in addition to skeletal myoblasts, cardiomyocytes, fibroblasts, and/or stem cells, one or more agents, including pharmaceutical carriers, antibodies, immunosuppressive agents, or angiogenic factors.

As already mentioned above, prior to introduction into a subject, the cells (especially when they are not autologous to the recipient subject) can be modified to inhibit immunological rejection. The cells can, for example, be rendered suitable for introduction into a subject by alteration of at least one immunogenic cell surface antigen. Additionally or alternatively, inhibition of rejection of transplanted cells can be accomplished by administering to the subject an agent which inhibits T cell activity in the subject. Such agents, or immunosuppressive drugs, include, but are not limited to, cyclosporin A, FK506, and RS-61443. The immunosuppressive drug may be administered in conjunction with at least one other therapeutic agent, for example a steroid (e.g., glucocorticoids such as prednisone, methyl prednisolone and dexamethasone) or a chemotherapeutic agent (e.g., azathioprine and cyclosphosphamide). An immunosuppressive drug is administered to a recipient subject at a dosage sufficient to achieve the desired therapeutic effect (e.g., inhibition of rejection of transplanted cells).

Dosage ranges for immunosuppressive drugs, and other agents which can be co-administered with these drugs, are known in the art (see, for example, B. D. Kahan, New Engl. J. Med., 1989, 321: 1725-1738). It is to be noted that dosage values may vary according to factors such as the disease stage, age, sex, and weight of the patient. Dosages can be adjusted to maintain an optimal level of the immunosuppressive drug in the serum of the recipient. Alternatively, immunosuppressive drugs may be administered transiently for a sufficient time to induce tolerance to the transplanted cells in the patient (see, for example, C. J. Green et al., Lancet, 1979, 2: 123-125; I. F. Hutchinson et al., Transplantation, 1981, 32: 210-216; B. M. Hall et al., J. Exp. Med., 1985, 162: 1683-1694; M. E. Brunson et al., Transplantation, 1991, 52: 545-549). Administration of the immunosuppressive treatment can begin prior (e.g., a few days) to transplantation of the cells into the subject. Alternatively, it can begin the day of transplantation or a few days (generally not more than three days) after transplantation. Administration of the immunosuppressive treatment is continued for sufficient time to induce donor cell-specific tolerance in the recipient such that donor cells will continue to be accepted by the recipient when drug administration ceases. Induction of tolerance to the transplanted cells in a subject is indicated by the continued non-rejection of the transplanted cells after administration of the immunosuppressive drug has ceased.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Some of the results presented below have been reported in two scientific publications (N. Dib et al., “Safety and Feasibility of Percutaneous Autologous Skeletal Myoblast Transplantation in the Coil-Infracted Swine Myocardium”, accepted for publication, J. Pharmacol. Toxicol. Methods, February 2006; and by P. I. McConnell et al., J. Thorac. Cardiovasc. Surg., 2005, 130: 1001.e1-1001.e12). These publications are incorporated herein by reference in its entirety.

### Example 1

### Correlation of Autologous Skeletal Survival with Changes in Left Remodeling in Dilated Ischemic Heart Failure

### Goals of the Study

Autologous skeletal myoblast (ASM) transplantation, or cardiomyoplasty, has been shown in multiple experimental studies to improve cardiac function after myocardial infarction (R. C. J. Chiu et al., Ann. Thorac. Surg., 1995, 60: 12-18; R. K. Li et al., Ann. Thorac. Surg., 1996, 62: 654-661; C. E. Murry et al., J. Clin. Invest., 1996, 08: 2512-2523; M. Scorsin et al., J. Thorac. Cardiovasc. Surg., 2000, 119: 1169-1175; K. Tambara et al., Circulation, 2003, 108 (suppl. II): 259-263; D. A Taylor et al., Nature Med., 1998, 4: 929-933; M. Jain et al., Circulation, 2000, 103: 1920-1927). Though the majority of studies have been performed in small animal models of myocardial injury, there is evidence of similar improvement in larger animal models (S. Ghostine et al., Circulation, 2002, 106(suppl. I): 131-136) and in the first patient trials (P. Menashé et al., Lancet, 2001, 357: 279-280; P. Menasché et al., J. Am. Coll. Cardiol., 2003, 41: 1078-1086; F. D. Pagani et al., J. Am. Coll. Cardiol., 2003, 41: 879-888). The mechanism behind such positive functional changes remains poorly understood given that developing and engrafted skeletal myoblasts are electro-mechanically isolated from their host myocardium, as evidenced by the lack of connexin-43 and/or gap junctions (M. Scorsin et al., J. Thorac. Cardiovasc. Surg., 2000, 119: 1169-1175; S. Ghostine et al., Circulation, 2002, 106(suppl. I): 131-136; P. Menashe et al., Lancer, 2001, 357: 279-280; P. Menasche et al., J. Am. Coll. Cardiol., 2003, 41: 1078-1086). Furthermore, clinical ASM cardiomyopathy has been applied exclusively to patients with severe ischemic cardiomyopathy, and more importantly, it has always been performed as an adjunct to coronary revascularization and/or left ventricular assist devices (LVDAs) (F. D. Pagani et al., J. Am. Coll. Cardiol., 2003, 41: 879-888). Because of these concomitant therapies, the improvements in indices of myocardial perfusion, viability and function may be difficult to attribute to ASM injection alone.

Additionally, growing experimental evidence suggests that the number of ASM cells transplanted and the functional/geometrical impacts are directly related (K. Tambara et al., Circulation, 2003, 108 (suppl. II): 259-263; B. Pouzet et al., Ann. Thorac. Surg., 2001, 71: 844-851). For example, Tambara et al. (Circulation, 2003, 108 (suppl. II): 259-263) using fetal-derived ASM in rats demonstrated that both cardiac function and remodeling were affected in a dose-dependent fashion. However, these benefits have not been demonstrated in ischemic dilated heart failure (HF), where elevated wall stresses and altered myocardial mechanoenergetics could compromise ASM survival, differentiation, and ultimately functional efficacy. Thus, the aims of the study reported herein were to evaluate LV remodeling and function after ASM transplantation into an animal model of end-stage ischemic HF (LVEF<35% and LV end-systolic volume>80 mL/m²). Furthermore, the study also sought to evaluate the survival, differentiation and alignment of ASM injected into those same animals.

### Materials and Methods

All experiments reported below were approved by The Ohio State University Institutional Laboratory Animal Care and Use Committed (ILACUC) and comply with published federal guidelines.

Ischemic Heart Failure Model:

Experimental ischemic heart failure was created in sheep as previously reported in dogs with minor modifications (H. N. Sabbah et al., Am. J. Physiol., 1991, 260: H1379-1384). Briefly, serial and selective left circumflex coronary artery (LCxA) microembolizations (2.9±0.4 injections per animal) were performed by injecting polystyrene beads (70-110 μm) weekly until the left ventricular ejection fraction was maintained at or below 35% for 2 consecutive weeks.

Experimental Groups.

The control Heart Failure group of sheep (baseline) was instrumented 2 weeks prior to LCxA microembolizations and heart failure induction (HF control, N=6). The transplanted group of cheep had LCxA microembolization and heart failure induction prior to instrumentation and injection with autologous skeletal myoblasts (HF+ASM, N=5). Studies were performed weekly for 6 weeks in awake and unsedated animals.

Chronic Instrumentation.

All sheep were instrumented through a left thoracotomy. A left ventricular solid-state electronic pressure transducer (4.0 or 4.5 mm, Konigsberg, Calif.) was placed into the left ventricle at its apex. Chronic, heparinized (1000 U/mL) fluid filled catheters (Tygon) were inserted for monitoring of aortic, left ventricular, and right ventricular pressures. Six piezoelectric crystals (Sonometrics Inc., New London, Ontario, Canada) were surgically placed in the left ventricle endocardium at the mild papillary level (short axis, SA), at the LV base and apex (long-axis, LA) and in the mid myocardium of the posterolateral LV (segment length, SLₚₒₛₜ). A 16 mm occluder (In Vivo Metrics, Healdsburg, Calif.) was positioned around the inferior vena cava (IVC). All catheters and cables were tunneled to positions between the animals' scapula.

Hemodynamic Measurements and Pressure Volume Analysis.

Aortic, right ventricular and left ventricular fluid filled catheters were attached to calibrated Statham pressure transducers (Model: P23XL; Biggo-Spectramed, Ocknard, Calif.) and amplified (Gould, Valley, Ohio). The electronic left ventricular pressure gauge was calibrated using the left ventricular fluid-filled catheter. Pressure waveforms were collected (at 1 kHz) and analyzed by a 16-channel data acquisition and software system (IOX; EMKA Techn., Falls Church, Va.).

Sonometric signals were analyzed for waveform cardiac-cycle dependent (end-diastolic and end-systolic) and independent (minimum, maximum, mean, etc) parameters. Left ventricular volume (mL) was calculated in real-time using short-axis (SA) and long-axis (LA) dimensions with the following equation:

LV volume=(SA²×LA²×π/6)/1000.

Left ventricular volume indices were calculated using the following equation:

LV volume indice=LV volume (mL)×body surface area (mL/m²).

Short IVC occlusions were performed for the generation of pressure volume relationships that were analyzed off-line analysis software (IOX, EMKA).

Left ventricular work was estimated (K. Todaka et al., Am. J. Physiol., 997, 272: H186-194; H. Suga et al., Physiol Rev., 1990, 70: 247-277) by calculating the pressure volume area (PVA). PVA was calculated from off-line end-systolic pressure volume relationship derived data as the sum of the left ventricle internal work (IW_{LV}) and stroke work (SW_{LV}).

IW_{LV}=(½[V₀−LVESV]×LVESP)

PVA=SW_{LV}+IW_{LV}

wherein V₀ is the volume of the left ventricle at zero pressure (x-intercept of Eₑₛ), LVESV is the left ventricle end-systolic volume (mL) and LVESP, is the left ventricle end-systolic pressure.

Skeletal Muscle Biopsy and Autologous Skeletal Myoblast Culture.

Skeletal muscle biopsy (1-3 grams) was harvested from the left forelimb of sheep at the time of the first microembolization in HF+ASM sheep. The forelimb muscle was exposed and the biopsy taken using sharp dissection avoiding electrocautery and placed into a tube containing biopsy transport media and shipped to GenVec, Inc. for autologous skeletal myoblast preparation and culture as previously described (M. Jain et al., Circulation, 2000, 103: 1920-1927).

All cells were expanded for 11-12 doublings and cryopreserved prior to transplant. The myoblasts were thawed, formulated in Transplantation Media, and shipped for direct myocardial injection. Myoblast purity was measured by reactivity with anti-NCAM mAb (CD56-PE, Cone MY-31, BD Biosciences, San Diego, Calif.) and by the ability to fuse into multinucleated myotubes. Cell viability was determined by Trypan Blue exclusion. Myoblasts were loaded into tuberculin syringes (˜1.0×10⁸ cells/mL) and shipped at 4° C. At the time of transplant, cells were allowed to warm slowly to room temperature, resuspended by gentle agitation and injected without further manipulation. Autologous skeletal myoblasts were injected at multiple sites in the infarcted myocardium in proximity to segmental sonomicrometry crystals.

Histology.

After six weeks, each animal was euthanized, the heart removed, and perfused with 10% buffered formalin. Tissue blocks were made from embolized myocardium receiving ASM injection. Hematoxylin & Eosin, and Trichrome stains were performed using standard methods.

Immunohistochemistry.

Deparaffinized sections were stained immunohisto-chemically with an anti-myosin heavy chain antibody that does not react with cardiac muscle, alkaline phosphatase-conjugated MY-32 mAb (Sigma, St Louis, Mo.), to confirm the phenotype of the mature grafts. Sections were developed with BCIP-NBT (Zymed Lab Inc., San Francisco, Calif.) and counter stained with nuclear red. Additionally stains for connexin-43 Ab (Chemicon, Temecula, Calif.), and cardiac specific troponin I (Chemicon) were performed.

Estimation of Myoblast Survival.

The heart was cut into blocks approximately 2.5 cm×2.5 cm×3 mm in dimension and processed in paraffin. In some cases, the whole block was sectioned (5 μm). In other cases, only a portion of the tissue was sectioned. For performing quantitative cell counts, tissue sections were then immunostained for skeletal-specific myosin heavy chain (MY-32). Using representative tissue sections and computer-assisted imaging analysis, the areas of engraftment were calculated and converted to the number of engrafted nuclei according to a separated count of nuclei density performed on Trichrome stained sections. The total number of surviving myoblast nuclei in each tissue block was calculated as:

(sum of graft area in section)×(density of nuclei per graft area)×(number of sections per block)×(Abercrombie Correction)

wherein the (number of sections per block) corresponds to the estimated number of sections per block according to approximated block thickness of 3 mm and section thickness of 5 and wherein the Abercrombie Correction is as described in M. Abercrombie, Ant. Rec., 1946, 94: 239-247.

Statistical Analysis.

Data are represented as mean±standard error of the mean (SEM). The differences between groups (treatments: HF+ASM and HF Control) over time for LV hemodynamic, geometric, and functional data were studied using multifactoral (two-way) analysis of variance (ANOVA) with repeated measurements (factors: group and time). If the F-ratio exceeded a critical value (alpha<0.05) the post-hoc Student-Newman-Keuls method as used to perform pair-wise comparisons (SigmaStat, Systat Software, Inc.).

Individual PV relationship were computed by regression analysis (IOX, EMKA Technologies). Additionally, the equality of the PV relationships between the HS+ASM and HF-Control groups was studied with multiple-linear regression considering both qualitative (group) and interaction terms; i.e., simultaneously testing the differences in slope and intersect of the regression functions (Minitab R14, Minitab Inc.). Linear regression analyses were also performed to study the relationship/interaction between indices of LV remodeling or function and the estimated number of surviving ASM-derived myocytes, including HF-Controls as zero survival (Minitab R14, Minitab Inc.).

In order to validate the impaired physiology of HF present in this model, the differences in the same control animals between pre- (baseline) and post-HF (week 1) were studied using a paired Student's t-test (SigmaStat, Systat Software, Inc.). However, since HF was defined as both, increased ESVI (LV end-systolic volume index) and decreased LVEF (LV ejection fraction) (a null hypothesis consisting of two variables), the Bonferroni method for multiple comparisons was used to correct the level of confidence (alpha <0.025).

### Results

Eleven sheep were studied for six weeks after establishment of heart failure with autologous skeletal myoblast injection (HF+ASM, N=5) or without (HF control, N=6). Three (3) of 8 sheep intended for the HF+ASM group died either during the instrumentation procedure; either before ASM injection (N=2) or within 72 hours after ASM injection, and therefore, were not included in the study. No sheep in the HF controls died early. Sheep were less active after HF induction, but no differences in daily observations were appreciated between groups.

Histology:

The average number of injected myoblasts was 3.44±0.49×10⁸ cells, ranging from 1.53 to 4.3×10⁸ cells. Myoblast purity, 92±1.4%, and cell viability, 93±1.2%, were assessed at the time of transport and myoblast viability was confirmed to be >90% (using trypan blue exclusion) after shipment (4° C.). ASM-derived skeletal myofibers were found in all injected hearts, but the estimated survival (see discussion below) of injected myoblasts surviving at week 6 ranged from 158,000 cells (0.05% survival) to 36.4 million cells (10.7% survival).

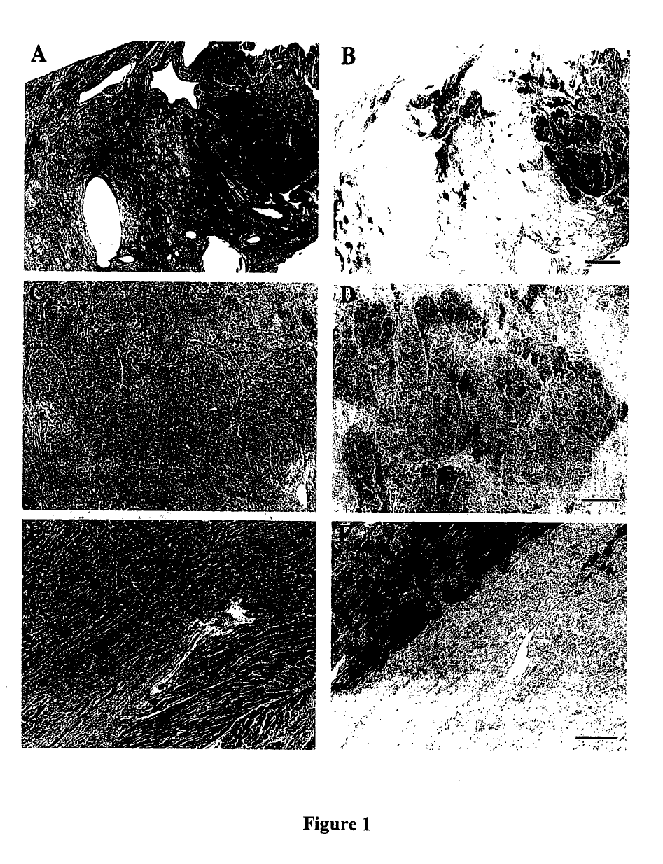

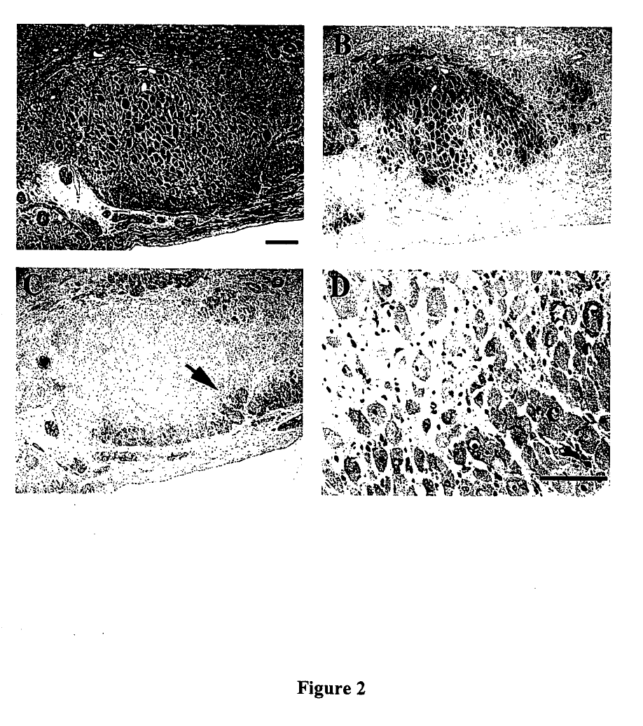

Representative histological sections with detailed descriptions are found in FIG. 1 and FIG. 2. In general, skeletal myocytes were seen aligned with other skeletal muscle fibers as well as aligned with remaining cardiac myocytes (FIG. 1C-F; FIG. 2 A, B). Engrafted skeletal muscle fibers were characterized by staining to the myosin heavy chain fast-twitch isoform (purple staining FIGS. 1 B, D and F and FIG. 2 B). However, in no section were ASM-derived myofibers seen stained for troponin I or connexin-43 despite close apposition to surviving cardiac myocytes (FIGS. 2 C and D, respectively).

Cardiac Hemodynamics:

Hemodynamic data are summarized in Table 1. No animal had improvement in dP/dTₘₐₓ (derivative of LV pressure) or LVEF after ASM injection. No linear relationship was found between the estimated number of surviving cells and LVEF (R²=0.00017, p=0.99) or dP/dTₘₐₓ (R²=0.048, p=0.543).

Pressure Volume Analysis:

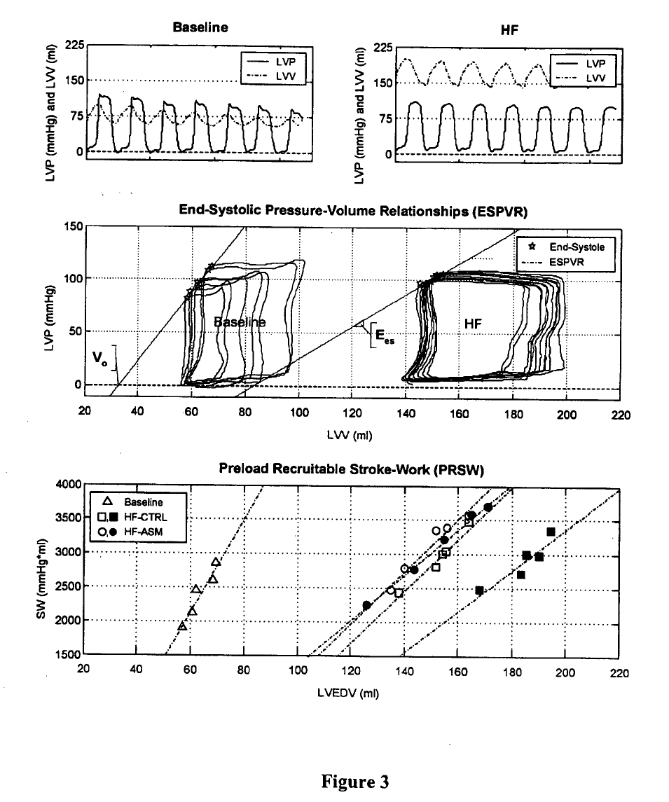

Data for ESPVR, PRSW and LV work (PVA) from HF controls and HF+ASM sheep are summarized in Table 1 and exemplified in FIG. 3. As expected and demonstrated by PV analysis, HF-induction resulted in significant decreases in the slope of Preload Recruitable Stroke Work (PRSW), M_{w}, and in the load-independent contractility index (Eₑₛ). No significant differences between treatment groups were observed at week 1, both presenting comparable degrees of dysfunction. Multiple linear regression analyses accounting for covariance between groups also demonstrated no significant differences in slope (WK1: p=0.614, WK6: p=0.519, power=1) or intercept (WK1: p=0.945, WK6: p=0.928, power=1) of the volume-adjusted PV-relationships. No linear relationship was found between the estimated number of surviving cells and Eₑₛ (R²=0.088, p=0.436) or M_{w} (R²=0.018, p=0.731).

There was an increase (rightward shift, p=0.026) in the V₀ (x-intercept) of the Eₑₛ for the HF controls from week 1 to week 6 (FIG. 3). The V₀ tended (p=0.20) to decrease (leftward shift) over the six weeks in the HF+ASM animals, and a difference was noted (p=0.014) between HF control and HF+ASM at week six, supporting that ASM injection attenuated LV remodeling. Likewise, the x-intercept of the PRSW (V_{w}) was increased from week 1 to week 6 in the HF control group (p=0.03), and remained different (p=0.009) as compared to the HF+ASM group at week 6 (Table 1 and FIG. 3).

Sonomicrometry and Left Ventricular Segmental Function:

Left ventricular regional and segment data are presented in Table 2. HF-induction significantly (P<0.05) increased the segmental length in the infarct region (SLₚₒₛₜ) of HF-control animals. Over the course of the study (week 1 to week 6), no significant differences were observed in SLₚₒₛₜ for either group of animals. Left ventricular segmental dyskinesia was present after microembolization, therefore, both systolic bulging (SB) and post-systolic shortening (PSS) were evident in both groups throughout the 6-week study.

Sonomicrometry and Left Ventricular Dimensions:

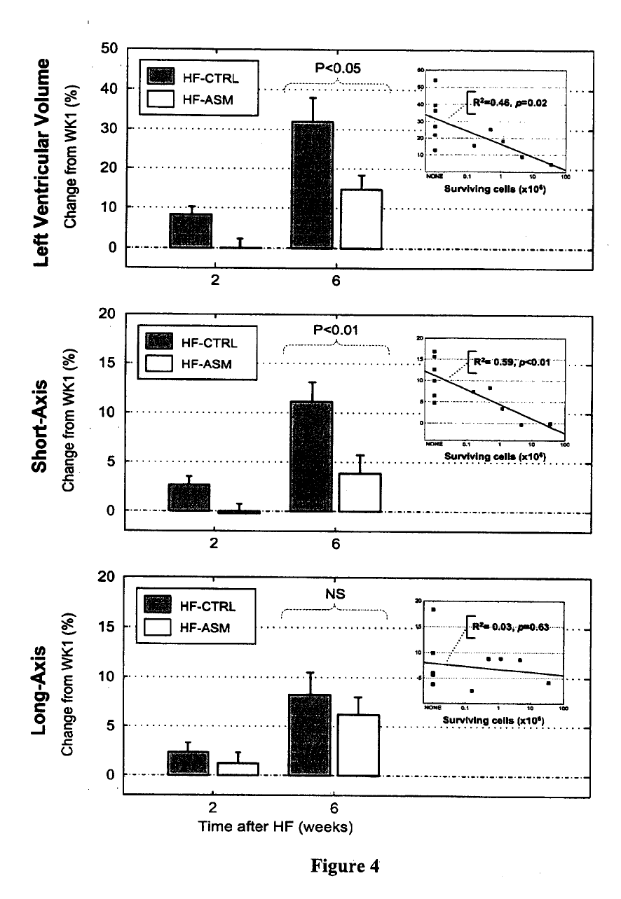

Left ventricular end-systolic and end-diastolic volume indexes (ESVI and EDVI, respectively) were increased (p<0.05) from baseline in both groups at HF week 1, however, there was no difference between groups at week 1 (Table 2). In HF+ASM, LV dilatation was attenuated as compared to HF controls (p=0.016) by week 3 (% change in ESVI: 5.3±1.2% and 17.8±3.3%, respectively) and this difference progressed (p=0.006) out to week 6 (FIG. 4). The difference in LV volume resulted from a significant (p=0.005) attenuation in SA dilatation alone (FIG. 4). No difference (P>0.5) was found in LA dilatation between groups. Correlations of ESVI, SA and LA to estimated ASM survival are presented in FIG. 4.

### Discussion

Few studies have examined the impact of ASM in hearts with a pre-existing and clinically significant degree of ischemia dysfunction and remodeling (LVEF <35% with LVESVI >80 mL/m²). The goal of the present study was to determine the therapeutic benefit of ASM cardiomyoplasty in a clinically applicable model of ischemic, dilated heart failure free of the confounding factors associated with coronary revascularization or other supportive therapies.

ASM-derived skeletal muscle was found in all injected sheep at six weeks. As others have reported (M. Scorsin et al., J. Thorac. Cardiovasc. Surg., 2000, 119: 1169-1175, 7-11; M. Jain et al., Circulation, 2000, 103: 1920-1927; S. Ghostine et al., Circulation, 2002, 106(Suppl. I): I131-136; P. Menasche et al., Lancet, 2001, 357: 279-280; P. Menasche et al., J. Am. Coll. Cardiol., 2003, 41: 1078-1083; F. D. Pagani et al., J. Am. Coll. Cardiol., 2003, 41: 879-888; N. Pouzet et al., Ann. Thorac. Surg., 2001, 71: 844-851), no staining for connexin-43 was found in ASM-derived skeletal muscle. Transplanted ASM-derived skeletal myofibers aligned with each other and with remaining cardiac myofibers in all sections (FIG. 1 and FIG. 2). Such organized alignment of the ASM-derived fibers suggests that these fibers remained sensitive to stress-strain relationships found within the myocardium (K. Kada et al., J. Mol. Cell Cardiol., 1999, 31: 247-259; M. A. Pfeffer and E. Bruanwald, Circulation, 1990, 81L 1161-1172; B. Z. Atkins et al., Ann. Thorac. Surg., 1999, 67: 124-129).

A major limitation with cell therapy, in general, is the large percentage (up to 90%) of cells that are lost shortly after injection (P. Menasche, Heart Failure Reviews, 2003, 8: 221-227; P. M. Grossman et al., Cardiovascular Interventions, 2002, 55: 392-397). An explanation for this early loss may be by means of lymphatic and/or venous drainage of the cells after direct intramyocardial (P. M. Grossman et al., Cardiovascular Interventions, 2002, 55: 392-397). Other factors also likely contribute to the further loss of cells that are retained within the myocardium/scar. Recently, investigations have shown that both the pre-treatment (M. A. Retuerto et al., J. Thorac. Cardiovasc. Surg., 2004, 127: 1-11) and transfection (A. Askari et al., J. Am. Coll. Cardiol., 2004, 43: 1908-1914) of ASM with VEGF improved cardiac function, presumably by enhancing perfusion and nutrient delivery. Furthermore, strategies to both limit inflammation and/or apoptosis have also proven beneficial to improving the efficacy after cellular cardiomyoplasty (Z. Qu et al., J. Cell Biol., 1998, 142: 1257-1267; M. Zhang et al., J. Mol. Cell. Cardiol., 2001, 33: 907-921). However, in the present study, evidence for inflammation was not observed at graft sites 6 weeks after injection (FIG. 1 and FIG. 2). Even with relatively low myoblast cell survival (FIG. 1, animal with 1.1% cell survival), considerable areas of scarred myocardium can be filled with viable myofibers as a result of cell fusion and subsequent enlargement of myofibers (approximately a 10-fold increase in ASM-derived myofiber cross-sectional area per nucleus versus myoblasts).

Left Ventricular Function:

Data evaluating cardiac performance after ASM injection in Tables 1 and 2 suggests no improvement in any hemodynamic parameter or in index of cardiac contractility in sheep with end-stage, dilated ischemic HF. The lack of a demonstrable direct functional benefit observed in the present study differs from reports in other animal models employing a single ischemic insult such as cryoinfarction (D. A. Taylor et al., Nature Med., 1998, 4: 929-933), ligation (M. Jain et al., Circulation, 2000, 103: 1920-1927), and coil embolization (S. Ghostine et al., Circulation, 2002, 106(Suppl. I): I131-136) and may be related to the chronic nature and severity of LV dysfunction in the present HF model (multiple microinfarctions over several weeks). Thus, in the sheep heart failure model, the insult may have more effectively exhausted remote myocardial compensatory mechanisms preventing contribution from the remote myocardium after ASM injection. This could explain the discrepancy with results previously published in sheep (S. Ghostine et al., Circulation, 2002, 106(Suppl. I): I131-136) after coronary occlusion.

Other possible explanations for the lack of observed functional cardiac improvements include the extent of remodeling at the time of treatment, the methods used for cell preparation, and technical flaws. In their ex vivo preparation in rats, Jain et al. (M. Jain et al., Circulation, 2000, 103: 1920-1927) noted that modest non-functional improvements observed after ASM injection were likely the result of benefits to non-functional properties of the LV, i.e., attenuated LV dilatation, rather than directly to LV contraction. In essence, less wall stress placed on remote cardiac myocytes as a result of ASM-derived skeletal muscle preventing further LV chamber dilatation would translate into better remote myocardial function. Perhaps the earlier the treatment the sooner the benefits of ASM-derived skeletal muscle could be realized on LV remodeling, and the greater the likelihood that the remote cardiomyocytes could adequately compensate and contribute to global LV function.

With respect to differences in the cell preparation in the present study, the limited functional benefit of the ASM in the present study could have resulted from the greater myoblast purity of the injectate, the method of cell expansion, cryopreservation, rewarming, and/or the transportation of the ASM. Unlike Pouzet and colleagues (B. Pouzet et al., Ann. Thorac. Surg., 2001, 71: 844-851), who demonstrated in rats stratified for LV function (LVEF) a significant correlation with the number of cells injected to indices of LV function; those most severely impaired received the greatest benefit, we were unable to demonstrate such as relationship compared to the number of surviving ASM-derived myocytes. Pouzet et al. (B. Pouzet et al., Ann. Thorac. Surg., 2001, 71: 844-851) and Ghostine et al. (S. Ghostine et al., Circulation, 2002, 106(Suppl. I): I131-136) present myoblast purity less than 50% at time of injection and showed improved systolic function, whereas we expanded a more pure population of myoblasts (>90% CD56 positive) and found none. Fibroblasts, as the major contaminant in these cell preparations, on their own have not been reported to enhance systolic function as has been reported for myoblasts (K. A. Hutcheson et al., Cell Transplant., 2000, 9: 359-368). However, synergistic effects between fibroblasts and myoblasts, which could account for improved contractility in preparations that are 50% versus 90% pure, cannot be ruled out.

Other obvious differences in the expansion and storage of cells in our study include the cryopreservation and subsequent thaw of cells prior to implantation, as well as shipment at 4° C. Histologically, we could not document any obvious differences in the contractile protein staining [MY-32], inflammation, ASM co-alignment and alignment with remaining cardiomyocytes from surviving grafts in our study as compared to those reported by others (D. A. Taylor et al., Nature Med., 1998, 4: 929-933; M. Jain et al., Circulation, 2000, 103: 1920-1927; S. Ghostine et al., Circulation, 2002, 106(Suppl. I): I131-136; B. Pouzet et al., Ann. Thorac. Surg., 2001, 71: 844-851). However, the overall effectiveness of cellular grafts is critically linked to various aspects of cell expansion, preservation and mechanisms of tissue retention, and therefore, are legitimate targets to explore to improve the efficacy of cellular cardiomyoplasty. Unfortunately, we did not directly study or vary myoblast purities or, for that matter, any other aspects of cell culture and preservation leading us to cautiously and intentionally avoided directly comparing the efficacy of one cell type or mixture versus another given the large difference in LV dysfunction and physiology in our report as compared the work of others (S. Ghostine et al., Circulation, 2002, 106(Suppl. I): I131-136; P. Menasche et al., Lancet, 2001, 357: 279-280; P. Menasche et al., J. Am. Coll. Cardiol., 2003, 41: 1078-1083; F. D. Pagani et al., J. Am. Coll. Cardiol., 2003, 41: 879-888; B. Pouzet et al., Ann. Thorac. Surg., 2001, 71: 844-851).

In addition, we cannot rule out the possibility that we did not adequately evaluate systolic function (Table 1), the time of study may have been insufficient or, there were simply an insufficient number of animals studied (the power of these studies was only sufficient to observe a 50% improvement in either LVEF or Eₑₛ). We believe based on our studies that with more severe LV dilation and dysfunction longer periods of time or perhaps larger dose of cells may be required for functional changes to be observed.

Left Ventricular Remodeling:

The major observation of the present study was the attenuation of LV dilatation after ASM transplantation (FIG. 4). Studies in both large and smaller animals have also shown positive effects on LV dilatation after ASM injection (Tambara et al., Circulation, 2003, 108 (suppl. II): 259-263; D. A Taylor et al., Nature Med., 1998, 4: 929-933; M. Jain et al., Circulation, 2000, 103: 1920-1927; S. Ghostine et al., Circulation, 2002, 106(suppl. I): 131-136, B. Pouzet et al., Ann. Thorac. Surg., 2001, 71: 844-851). However, a novel finding of the current study was that effects on LV dilatation were exclusive for the SA dimension. The mechanism(s) that defines this preferential effect on SA remodeling is not entirely clear. The idea that cellular cardiomyoplasty may be directly affecting scar elasticity and thereby limiting scar expansion is a possible explanation for attenuated regional dilatation (T. Jujii et al., Ann. Thorac. Surg., 2003, 76: 2062-2070). Although we could not find a measurable improvement in either post-systolic shortening or systolic bulging after ASM injection, the interplay of both in chronically ischemic myocardium has not been well characterized (H. Skulstad et al., Circulation, 2002, 106: 718-724).

If ASM-derived skeletal myofibers can actively resist forces (stretch) inline with their fibers, as demonstrated ex vivo (C. E. Murry et al., J. Clin. Invest., 1996, 08: 2512-2523), and thereby limit LV dilatation, this might also explain the observed attenuation to LV dilatation selectively for the LV short axis. For example, as the ventricle becomes increasingly spherical after ischemic injury, the predominant cardiac fiber axis (e.g., 60°) progressively re-orients towards the horizontal or short-axis (e.g., 30°) (F. Torrent-Guasp et al., Semin. Thor. and Cardiovasc. Surg., 2001, 13: 298-416). In the present study, ASM-derived skeletal myofibers were found to be aligned with each other and with remaining cardiac myocytes and therefore, theoretically, the engrafted ASM-derived myofibers' orientation would be more aligned with the LV short axis. As suggested by our data in a small number of animals, ASM-derived myofibers may offer innate resistance to dilatory forces upon or along their fiber lengths, thereby, selectively preventing dilatation aligned with ASM engraftment along the LV short axis (FIG. 4).

Study Limitations:

The animal model used in the present study approximated clinical ischemic heart failure in etiology, degree of pathology and coronary anatomy (H. N. Sabbah et al., Am. J. Physiol., 1991, 260: H1379-1384; M. A. Pfeffer and E. Brunwald, Circulation, 1990, 81: 1161-1172; P. Menasche, Heart Failure Reviews, 2003, 8: 221-227). Microembolization does not fully model the phenomenon of myocardial infarction leading to ischemic HF in all patients, particularly those patients who suffer a single large infarct. Moreover, this model greatly accelerates the disease progression typical for chronic ischemic HF (M. A. Pfeffer and E. Brunwald, Circulation, 1990, 81: 1161-1172; M. A Pfeffer, Annu. Rev. Med., 1995, 46: 455-466).

Each animal underwent the same number and types of procedure's. Differences found between the groups in the present study could have resulted of the timing of instrumentation (and ASM injection). The fact that attenuated dilatation was observed only in the SA dimension in HF+ASM animals, while LA dilatation was nearly identical between the HF control and ASM groups, supports that differences observed between the groups could have been dependent upon myoblast injection. We have attempted to provide a best estimate of cell survival using standardized techniques to quantify the number of viable ASM-derived myocytes at 6 weeks so that the relative survival between animals could be compared; however, significant sampling error can exist in the method used to calculate cell survival (M. Abercrombie, Ant. Rec., 1946, 94: 239-147). Therefore, values given for cell survival should not be interpreted as absolute, but only as a standardized estimate.

Segmental and/or regional function as measured by sonomicrometry may have not adequately documented function in the exact area of ASM engraftment due to the variability of ASM survival; however, myoblast injection was specifically targeted to and was found in the immediate vicinity of the sonomicrometry crystals at 6 weeks. Left ventricular function in the awake animal preparation used in this study, as evaluated via pressure volume analyses, was not able to be performed at extremely low ventricular volumes due to autonomic activation and inevitable adverse hemodynamic consequences. Therefore, the estimates of slope for Eₑₛ and M_{w} do not include low volume measurements. If the methodology existed in awake animals to permit an evaluation of function over a wider range of preloads, as possible with an isolated heart preparation (M. Jain et al., Circulation, 2000, 103: 1920-1927), the possibility exists that a difference could have been found in both position and slope of these relations.

### Conclusions

The study presented as Example 1 describes ASM transplantation in a clinically applicable large animal model of chronic ischemic HF free of concomitant interventions. Despite the apparent lack of direct functional impact on cardiac function in this small group of animals, we were able to demonstrate a significant attenuation in LV dilatation after ASM transplantation. The attenuation in LV dilatation was exclusive to the short axis and correlated with an estimate of surviving ASM-derived myocytes. These observations suggest that ASM affect LV remodeling by a mechanism independent of cell-to-cell communication and/or direct functional improvements, but that ASM engraftment and alignment do play a role in such a mechanism.

### Example 2

### Correlation of Autologous Skeletal Survival with Changes in Left Remodeling In Dilated Ischemic Heart Failure: Contribution of the Remote Vs the Transplanted Myocardium

Introduction.

Autologous skeletal myoblast (ASM) injection after myocardial infarction has been shown to improve left ventricular (LV) performance. However, the mechanism(s) behind such improvement remain(s) unclear.

Methods.

Ischemic heart failure (iHF) was induced in sheep (N=12) by selective microembolizations (circumflex artery). After iHF (LVEF: 33±2.2%; LVESV: 143±18 mL), animals were instrumented with sonomicrometers to assess global and segmental LV function. The infarcted myocardium (INF) was injected with either 5×10⁸ cells (ASM; N=6) or cell media (CM; N=6). Pressure volume analyses, hemodynamics and LV segment function (both INF and remote/anterior myocardium [RMT]), were evaluated weekly in unsedated animals for 10 weeks. Comparisons were made by 2-way ANOVA.

Results.

ASM-derived myofibers were found histologically in all ASM animals. There were no differences between groups in any parameter at 1 week. LV remodeling was attenuated in ASM vs CM (change LVESV week 1 to week 10: 17.8±5.8 mL vs 55.4±9.8 mL; p<0.001); while improvements in LVEF (change week 1 to week 10: 5.6±1.1% vs 0.51±1.3%; p=0.002) and preload recruitable stroke work (Mw, change week 1 to week 10: 21±6.3 vs −11.2±6.3; p<0.001) were found after ASM. INF systolic fractional shortening (sFS, 0.8±1.1%) was not improved after ASM or CM (change week 1 to week 10: 0.87±0.53% vs −0.07±1.43%; p=0.44). However, RMT sFS (18.3±1.1%) was improved after ASM vs CM (change week 1 to week 10: 3.0±1.0 vs −1.93±0.54%; p<0.001).

### Conclusions

ASM-derived myofibers promoted attenuation in LV remodeling, improved LV function and uniquely, more effective remote [non-infarct] myocardial compensation/function. Therefore, ASM transplantation earlier after myocardial infarction may provide for better improvements in LV remodeling and contractility.

### Example 3

### Safety and Feasibility of Percutaneous Autologous Skeletal Myoblast Transplantation in the Coil-Infarcted Swine Myocardium

All experiments were conducted according to guidelines published in the “Guide for the Care and Use of Laboratory Animals” (DHHS publication number NIH 85-23, revised 1985) and Subchapter A of the Federal Animal Welfare Act written by the United States Department of Agriculture and in the spirit of FDA Good Lab Practices. The study protocol was approved by the Harrington Animal Care and Use Committee at Arizona Heart Hospital, Phoenix, Ariz., prior to the start of the study. A summary of the study design is shown in Table 3.

### Materials and Methods

Animal Preparation.

Ten (10) female Yorkshire swine between the ages of 3 and 6 months and weighing 91±25 lbs, underwent induced myocardial infarction. Three (3) died during or shortly after induction of the myocardial infarction. One (1) animal was used to evaluate short term retention and biodistribution of injected myoblasts, and six (6) animals served as recipient animals for either ASM or transport medium only.

Immediately prior to inducing infarction, Electrocardiograghy (EKG) Echocardiography, cardiac output and index, and blood values were assessed. Each animal was anesthetized with intramuscular Telozol (tileamine hydrochloride and zolazepam hydrochloride; 500 mg), intubated, and mechanically ventilated with 2% isoflurane and 3-L/min oxygen. An 8-F arterial sheath was inserted into the right femoral artery using either percutaneous or cutdown technique, and selective left and right coronary angiography, left ventriculography and NOGA™ mapping were performed.

Concurrent with the femoral cutdown, a skeletal muscle biopsy was taken from each of the seven (7) studied swine. Under sterile conditions, a 6-cm incision was made longitudinally along the right hind limb, and a 5-10 grams of muscle from the thigh muscle was removed with a sharp dissection technique. The incision was closed in layers. The muscle biopsy was placed immediately in a biopsy transportation medium on ice and sent to a cell culturing facility for myoblast expansion.

Following the muscle biopsy, an implantable loop recorder (ILR) was inserted in each swine. The ILR use was the Medtronic Reveal®Plus 9526 (Medtronic, Minneapolis, Minn.), a single-use programmable device designed to continuously record a subcutaneous electrocardiogram (ECG) during arrhythmic events. Using a sterile technique, a single 2 cm incision was made along the left side of the spine just above the heart level. The wound was dissected to the fascia, and an approximated 4×2 cm subcutaneous pocket was formed over the muscle. The event monitor was placed subcutaneously, and the ECG signal quality and amplitude were verified. Wound closure was performed in a conventional fashion.

Infarction Model.

Immediately following ILR implantation and left heart catheterization, an anterior infarction was induced in each of the seven swine by coil embolization using either a 2×10 mm complex helical or a 3×23 mm diamond shape Vortx coil (Boston Scientific/Target, Natick, Mass.) to the distal left anterior descending (LAD) artery. Coronary occlusion occurred in an average of 16 minutes after coil deployment, as demonstrated by coronary angiography and ECG showing ST elevation in V₁-V₃ a few minutes after occlusion of the left anterior descending artery. The femoral artery was closed with either an Angio-Seal vascular closure device or using sutures, and the animals were recovered per standard operating procedures. Significant ventricular arrhythmias were treated with a 2% intravenous lidocaine bolus and electrical cardioversion. Post-procedural discomfort was treated with intramuscular butorphanol tartrate (Dolorex, 1.0 mL).

Expansion of Myoblasts.

The autologous skeletal myoblasts were isolated by fine mincing of the muscle tissue followed by a three step enzymatic digestion containing a 0.5 mg/mL trypsin (Invitrogen, Carlsbad, Calif.) and 0.5 mg/mL collagenase (Crescent Chemical Co., Islandia, N.Y.). Cells released in each step were washed and plated on gelatin coated dishes. The cells were expanded over two passages in a growth medium (GM) composed of SkBM® (Skeletal Muscle Basal Medium, Cambrex Corporation, Walkersville, Md.) supplemented with 15% (vol/vol) fetal bovine serum (Hyclone, Logan, Utah), 10 ng/mL rhEGF (Cambrex), 3.9 μg/mL dexamethasone (American Reagent Lab, Shirley, N.Y.), and 50 μg/mL gentamicin (Invitrogen). The cells were maintained at less than 70% confluence to prevent spontaneous cell fusion, and were harvested by trypsin/EDTA digestion (Invitrogen) and cryopreserved. For the long-term survival study, approximately 10% of the culture was labeled with bromodeoxyuridine (BrdU) during the last 24 hours of culture to aid histological identification of the transplanted cells.

In preparation for cell injection, frozen myoblasts were thawed and washed twice in growth medium and twice in transplantation medium. Finally, the cells were brought to the proper cell density, into 1 mL syringes and shipped either on ice or cold packs to the animal study facility.

To label cells with iridium, 40×10⁶ cells from the animal were mixed with 13.4×10¹⁰ iridium particles (0.3 μm diameter, supplied by BioPhysics Assay Laboratory, Worcester, Mass.) and incubated for 1.5 hours at 37° C. to foster internalization of iridium by the myoblasts. Non-internalized particles were removed by washing the cells six times in growth medium. The remaining labeled cells were mixed with unlabelled myoblasts to formulate the final cell product in transplantation medium and loaded in 1 mL syringes. Aliquots of the final cell product were retained so that a standard curve could be generated (see below).

Characterization of Cell Population.

Cells were analyzed for viability, sterility, purity, and potency. Viability was assessed using trypan blue, and sterility was measured using a membrane filtration method. The LAL (Limulus Amebocyte Lysate) Gel clot assay to detect endotoxins. Cell purity was determined by FACS (Fluorescence Activated Cell Sorting) using a primary antibody against myoblast-specific α7-integrin (H36 provided by Dr. Kaufman, University of Illinois). Myoblast potency was assessed using a fusion assay performed by switching confluent myoblast cultures to fusion media. Under these conditions, myoblasts fuse and form multinucleated myotubes. Contaminating fibroblasts do not have this property and remain as single cells.

At the time of final formulation in myoblast transplantation media, the cell viabilities were between 60% and 96% (see Table 4). Upon receipt of the myoblasts at the animal study site, the viabilities had decreased. The purity of the cell preparations ranged from 30% to 62%, with contaminating cells possibly being fibroblasts. All transplanted cells passed USP filtration sterility and endotoxin LAL testing.

Autologous Myoblast Transplantation.

Prior to initiating implantation studies using the MyoStar™ Intramyocardial Injection Device (BioSense-Webster, Diamond Bar, Calif.), preliminary biocompatibility studies were performed. Similar to a myogenic cell line (U. Oron et al., Int. J. Cardiovasc. Intervent., 2000, 3: 227-230), the data showed no significant alteration in cell number or cell viability after passing through the catheter at a range of cell concentrations from 10×10⁶ to 100×10⁶ cells/mL (data not shown). Approximately thirty (30) days after infarction, ASM were transplanted into each of the treatment swine. Each animal was anesthetized as described earlier. An 8-F arterial sheath was inserted into the left femoral artery using a cutdown technique and myocardial assessments were repeated.

Percutaneous autologous skeletal myoblast transplantation was performed using an 8-F arterial sheath to advance the MyoStar™ Intramyocardil Injection Device through either the right or left femoral artery. A 3-D unipolar voltage map (NOGA™) was used to determine the area of infarction and to guide the needle-injection catheter. An average of 137 points were used to map the left ventricle, and a mean unipolar voltage of 7.8±1.5 mV (bipolar: 2.3±0.4 mV) was used to detect infarcted areas (D. J. Callans et al., Circulation, 1999, 100: 1744-1750). The catheter used was either a B or C. The injection needle was measured in the straight and curved positions (90 degrees) and adjusted to extend 3 to 5.5 mm into the infarcted region of the endocardium depending on the wall thickness measured by echocardiography. Penetration was verified by either fluoroscopy, ST elevations, or premature ventricular contractions during needle advancement.

Immediately prior to injection, each syringe was warmed to room temperature and inverted several times to ensure a homogeneous cell suspension. The temperature was assessed by touch and homogeneity was assessed visually. The suspended cells were injected into the center and peripheral edges of the infarcted region of the myocardium. Group 2 animals received ˜300×10⁶ cells, and Group 3 animals received ˜600×10⁶ cells. Group 1 control animals were injected with myoblast transplantation media using similar numbers of injections and injection volumes. Table 5 describes dosing characteristics in detail. After the injections were complete, the femoral artery was closed with either an Angio-Seal vascular closure device or sutures, and the animals were recovered.

Quantitation of Distribution of Iridium-Labeled ASM.

Two (2) hours after the final injection, the animal injected with iridium labeled ASM was sacrificed and the heart, brain, kidneys, liver, lungs and spleen were weighed. The anterior, lateral, inferior and septal regions of the left ventricle were cut into eight equal segments (two vertical segments for each region, and 5-9 g. of each organ was removed for analysis. All tissue samples and labeled cell standards were placed in vials and dried overnight at 70° C.

The resulting dried samples were sent to BioPhysics Assay Laboratory for analysis involving two steps: activation and detection. During activation, the samples were exposed to high-energy neutrons allowing the iridium atoms in the cells to capture incident neutrons. The unstable radioactive products of the neutron flux were then allowed to decay for two days to reduce background interference. During the detection phase, the samples were placed in a high-resolution gamma-detection monitor that measured the energy level and the number of gamma particles emitted. A standard curve generated from samples containing known numbers of iridium-labeled cells was used to convert the gamma particle emission for each tissue sample to the number of retained iridium-labeled cells. To calculate the total number of labeled cells within each whole organ (other than the heart), the value for each tissue sample was multiplied by the weight of the organ divided by the sample tissue weight.

Safety Assessments.

Safety was evaluated by animal survival, well-being, heart rhythm, blood tests and adverse events. Well-being and survival were continuously monitored and recorded during the 90-day study period. Heart rhythm was monitored using a standard 12-lead electrocardiogram, obtained in a resting, supine position at selected time-points, and by an implantable loop recorder (ILR). The ILR was activated during, and 24 hours following myocardial infarction and transplant. Additional interrogations were performed 3 times per week for 2 weeks after each procedure and weekly between transplant and harvest. Each device was programmed as follows: Storage mode—13 auto-activated events for 42 minutes to detect bradycardia <30 bpm, tachycardia >230 bpm, asystole >3 seconds for 16 consecutive beats. All ILR devices were set for a maximum gain of 8 (±0.2) mV and sensitivity was adjusted between 10 and 13 to achieve optimal sensing.

Hematology and Chemistry.

Hematology and chemistry specimens were drawn at each intervention after the animals were fasted overnight. Blood was collected from the femoral access under anesthesia.

Myocardial Function Assessment.

Functional assessment of the hearts was performed at selected time points to compare the effects of cell transplantation from baseline and to compare the treated animals with controls.

Ejection fraction and ventricular wall thickness were assessed using a standard resting echocardiogram (ECHO). The ECHO was 2-dimensional and performed in the parasternal long and short axis views, four, two and long axis apical and subcostal four and short axis views. ECHO results were interpreted in a blinded fashion by an experienced cardiologist. Additional ejection fraction assessments were made by ventriculography (LV gram). Coronary arteries were visualized for patency through coronary angiography during left heart catheterization using the right and left anterior oblique projections, and were interpreted by the investigator. Cardiac index was assessed by non-invasive impedance cardiography (ICG) using a BioZ device (Cardio Dynamics International Corporation, San Diego, Calif.). Four (4) ICG sensors were attached to each animal (one on both sides of the neck and torso), and a correction factor of 1.48 was used to adjust the values for pig chest anatomy (C. J. Broomhead et al., Br. J. Anesth., 1997, 78: 323-325). Three-dimensional electromechanical mapping was performed using the NOGA Biosense Navigational System (Biosense Webster, Diamond Bar, Calif.) via a 7-F NAVI-STAR™ catheter advanced through the 8-F sheath into the left ventricle. The mapping was used to identify areas of normal tissue, ischemia and infarction of the ventricle, as described previously (R. Kornowski et al., Circulation, 1998, 98: 1116-1124). These maps included unipolar and bipolar voltage maps which were used to calculate left ventricular unipolar voltage (LVUPV), apical unipolar voltage (APUPV), left ventricular bipolar voltage (LVBPV) and apical bipolar voltage (APBPV). A number and color scale to indicate the voltage in each area of the myocardium were assigned by the computer.

Histology.

Histological analysis was performed on all hearts from the treatment and control group animals following harvest at day 90 of the study. The hearts were weighed and preserved in 10% neutral-buffered formalin. The infarcted portion of each heart was embedded in paraffin, sectioned and mounted on slides, which were stained to identify presence of cellular engraftment and inflammatory reaction to the procedure. Histological stains included Hematocylin & Eosin, and Trichrome. Immunohistochemical stains included skeletal muscle-specific myosin heavy chain (MY32), and immunoreactivity to bromodeoxyuridine (BrdU).

### Results

Retention and Biodistribution.

The retention of myoblasts in the selected tissues 2 hours following catheter-based injection into the myocardium is listed in Table 4. No iridium-labeled ASM were detected in the brain, kidney or liver. Very low numbers of cells were detected in the spleen and in areas of the left ventricle not targeted for cell injection (<0.1% of the injected cells). Two adjacent myocardial regions, which were the targets of the injections, contained the majority of the cells retained in the heart. In total, 4.1% of the injected cells were detected in the apical region of the heart that contained the scar tissue. The primary site of outside of the heart where cells were detected was the lung which contained 5.1% of the injected cells.

Safety.

Injections of control media or cells for determining safety and effects on myocardial function were performed as summarized in Table 5. In all groups, there were no complications or deaths related to the catheter-based delivery of ASM. No significant differences in hematology and blood chemistry were seen between the two treatment groups and controls at any selected time point (data not shown). In addition, no arrhythmias were recorded by ILR in any group during the 60-day period following ASMT. One episode of non-sustained ventricular tachycardia and 2 episodes of sinus tachycardia were recorded, all three prior to transplantation.

Myocardial Function.

Functional assessments of the hearts were performed to detect and compare changes in viability and function that may have occurred in the treatment groups compared to controls. At the time of transplant (baseline), no significant differences in EF by ECHO, EF by LV gram, cardiac index by Bio-Z, left ventricular unipolar voltage (LVUPV) by NOGA, and apical unipolar voltage (APUPV) by NOGA were found between the treatment and control groups.

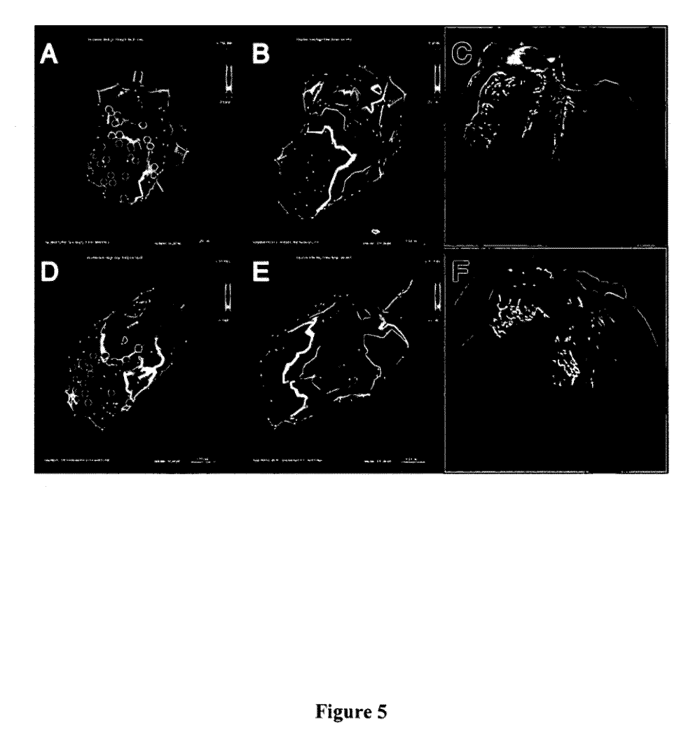

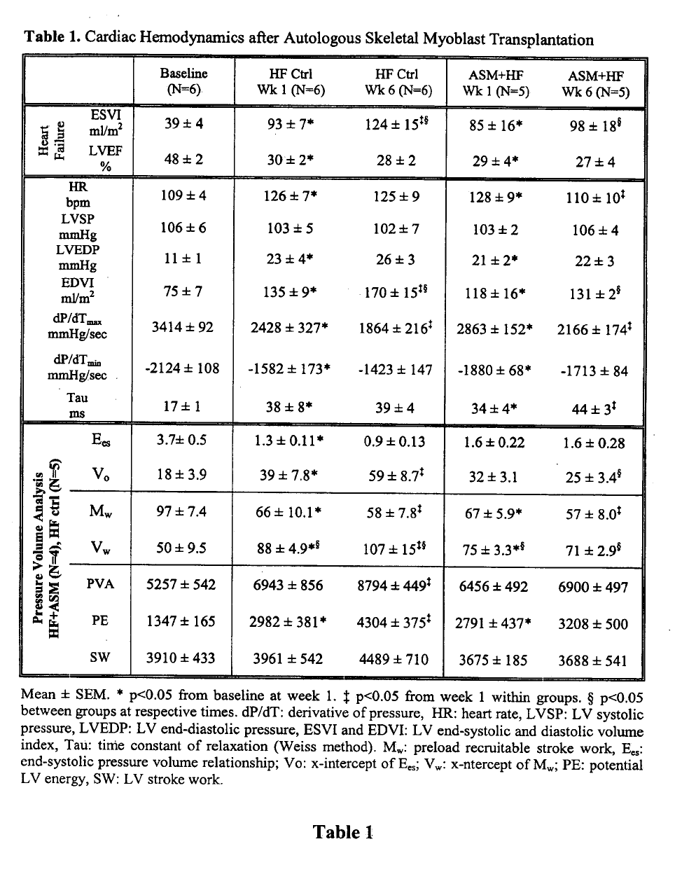

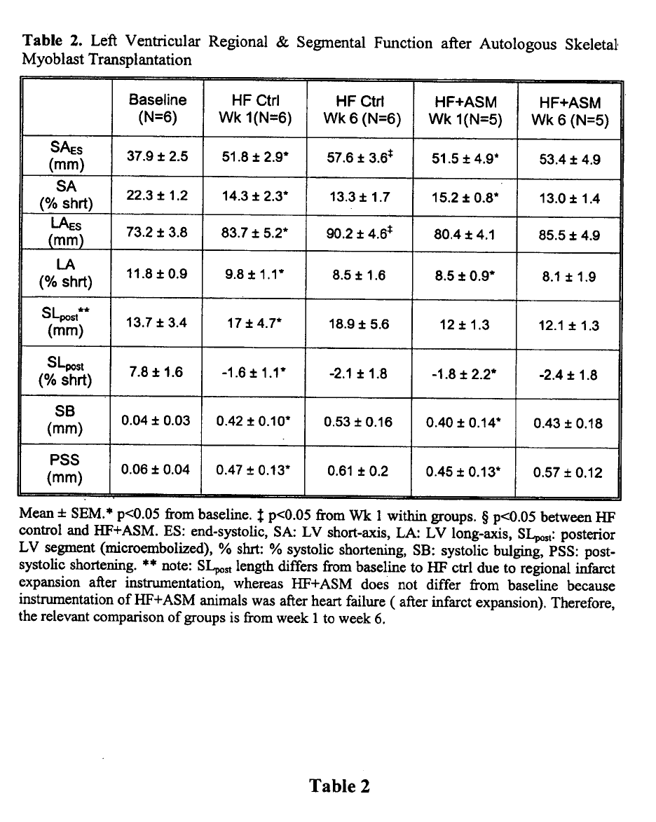

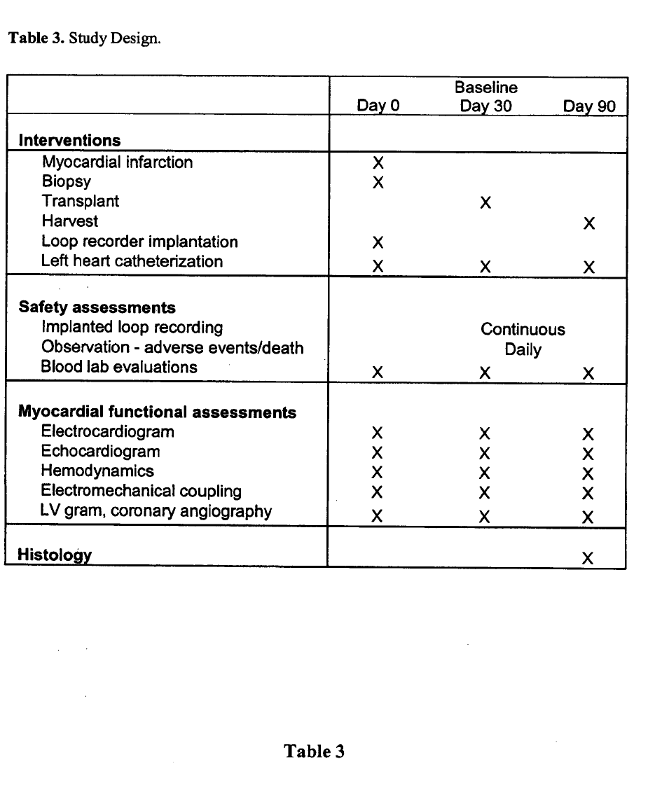

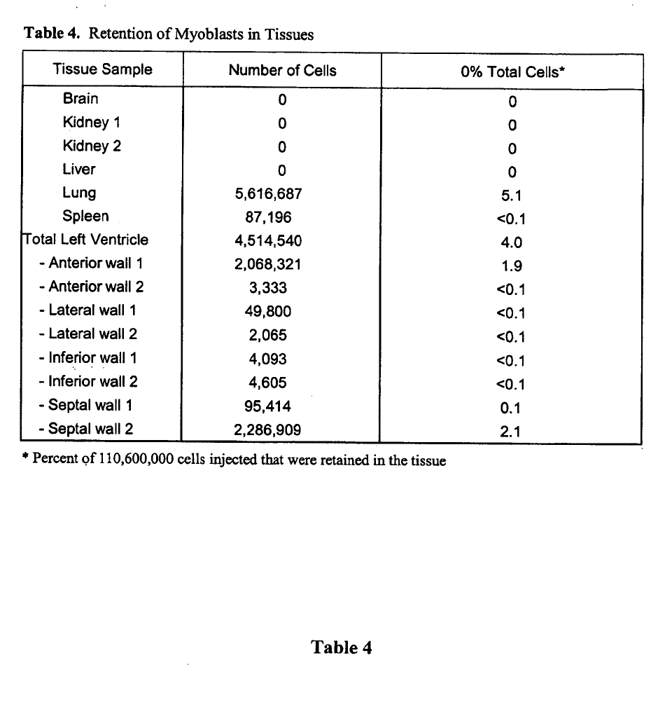

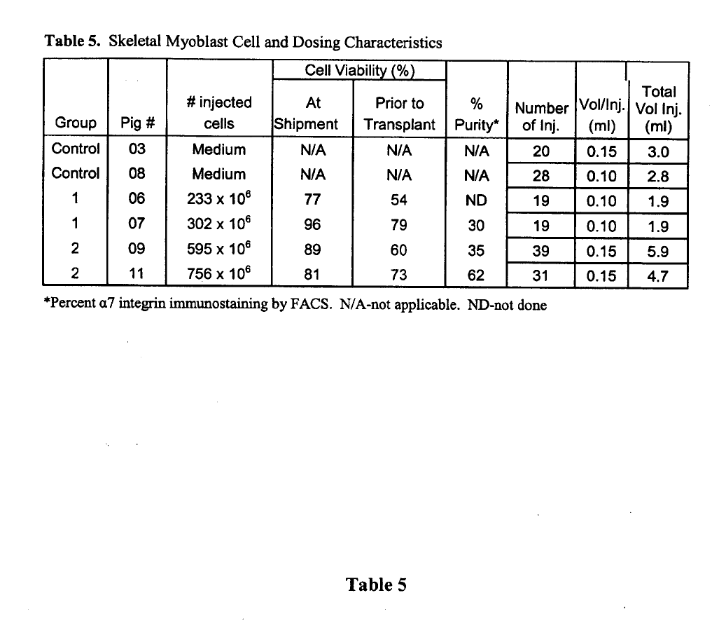

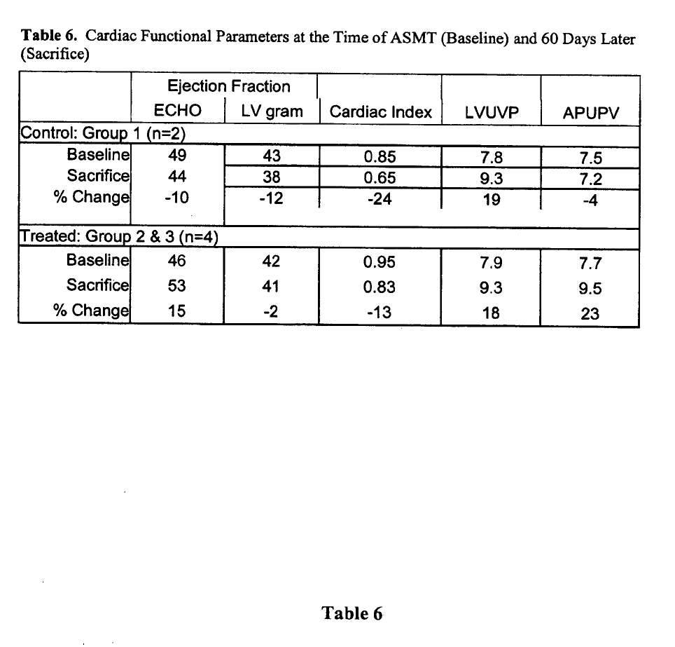

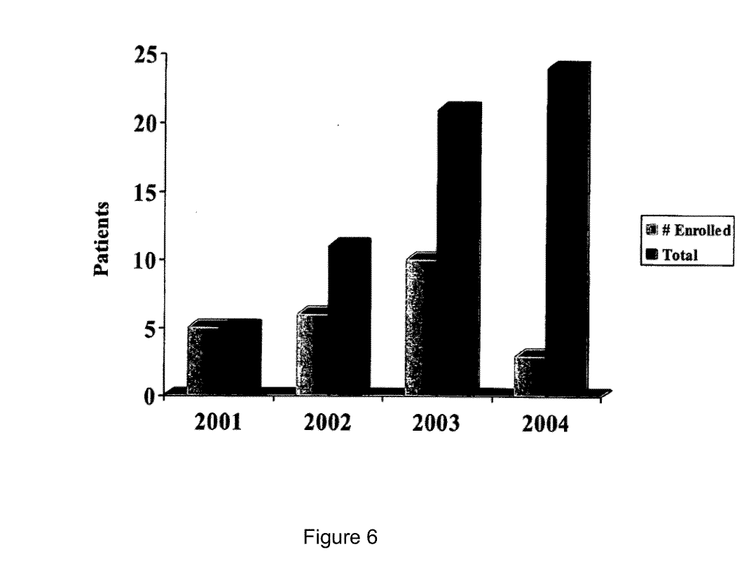

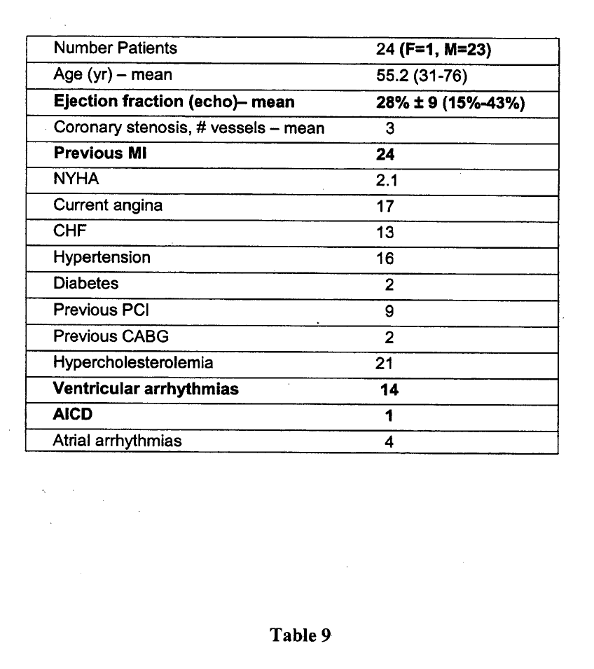

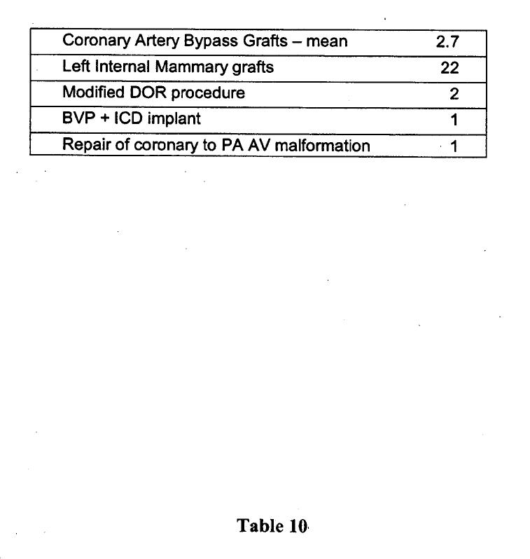

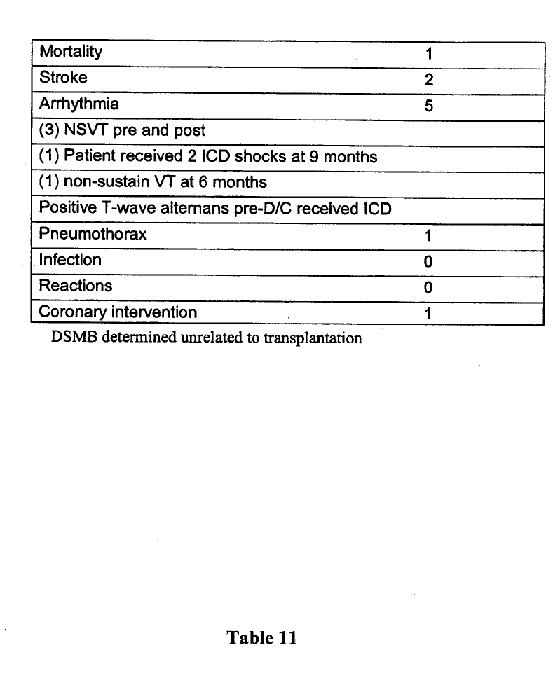

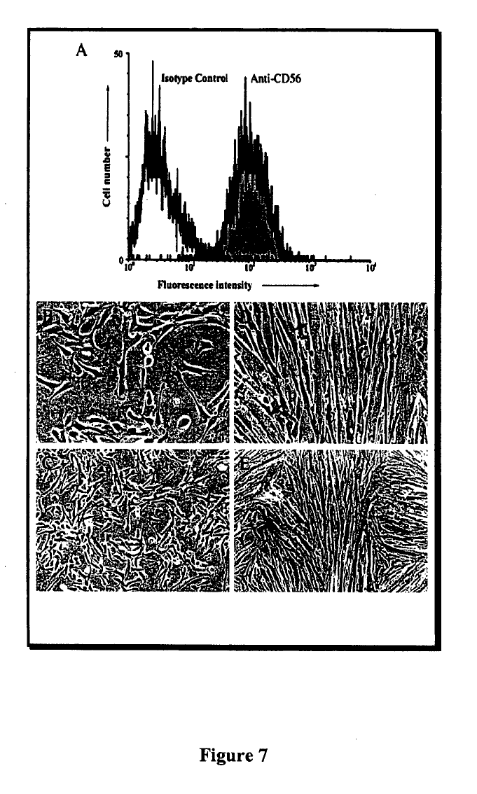

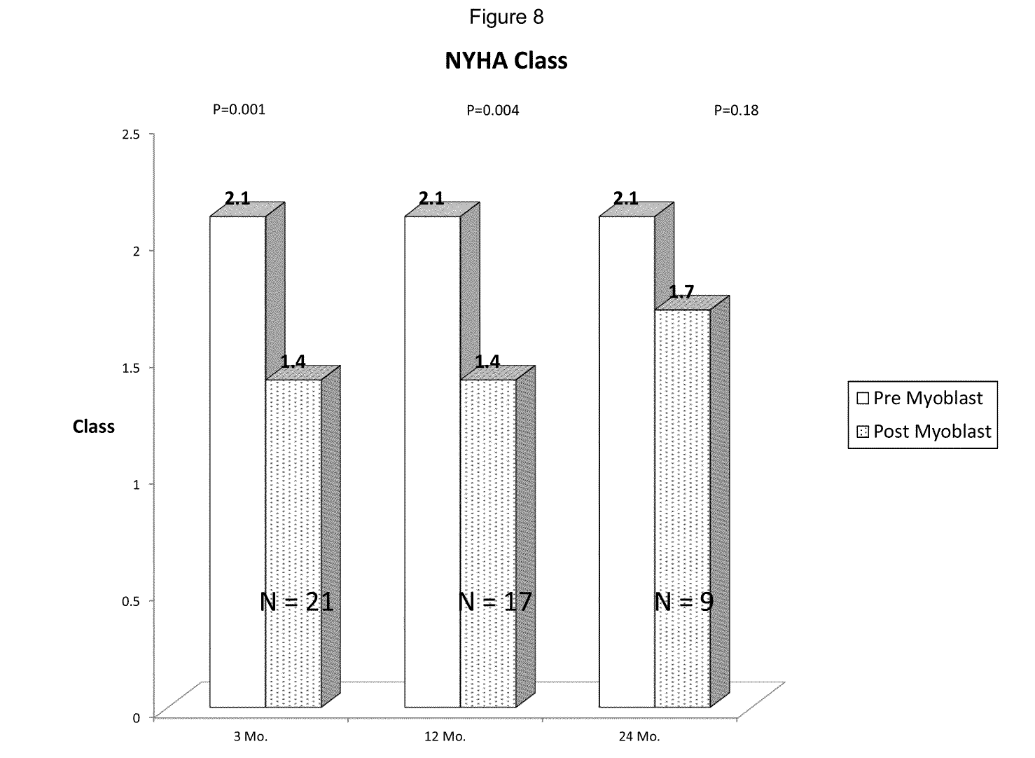

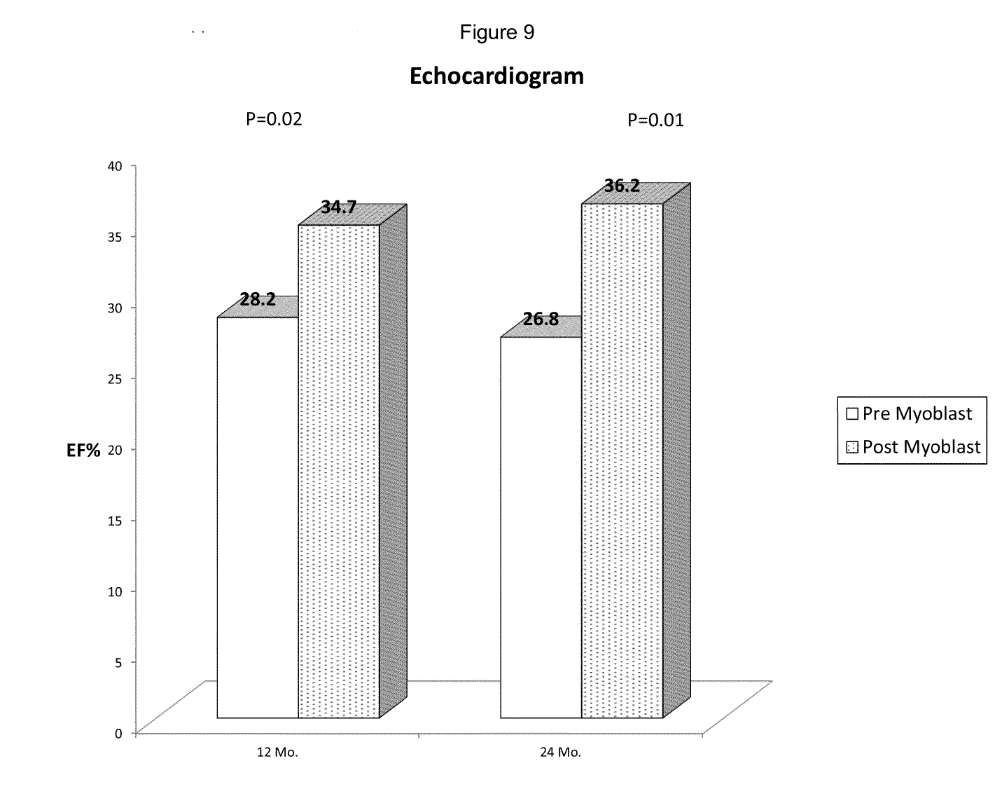

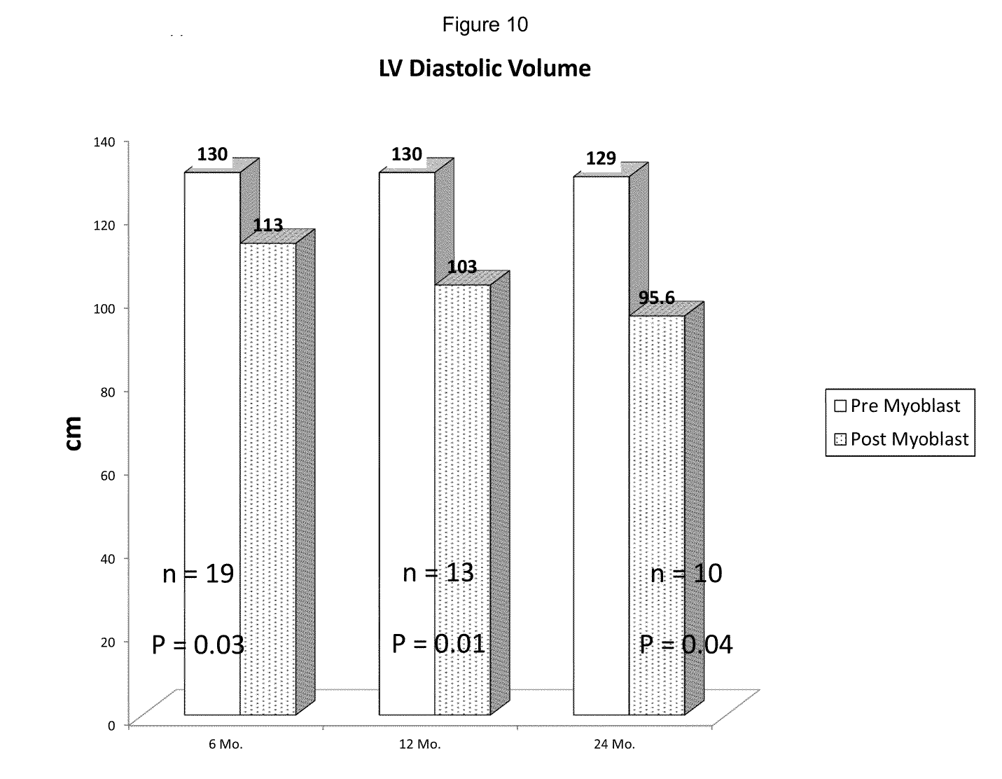

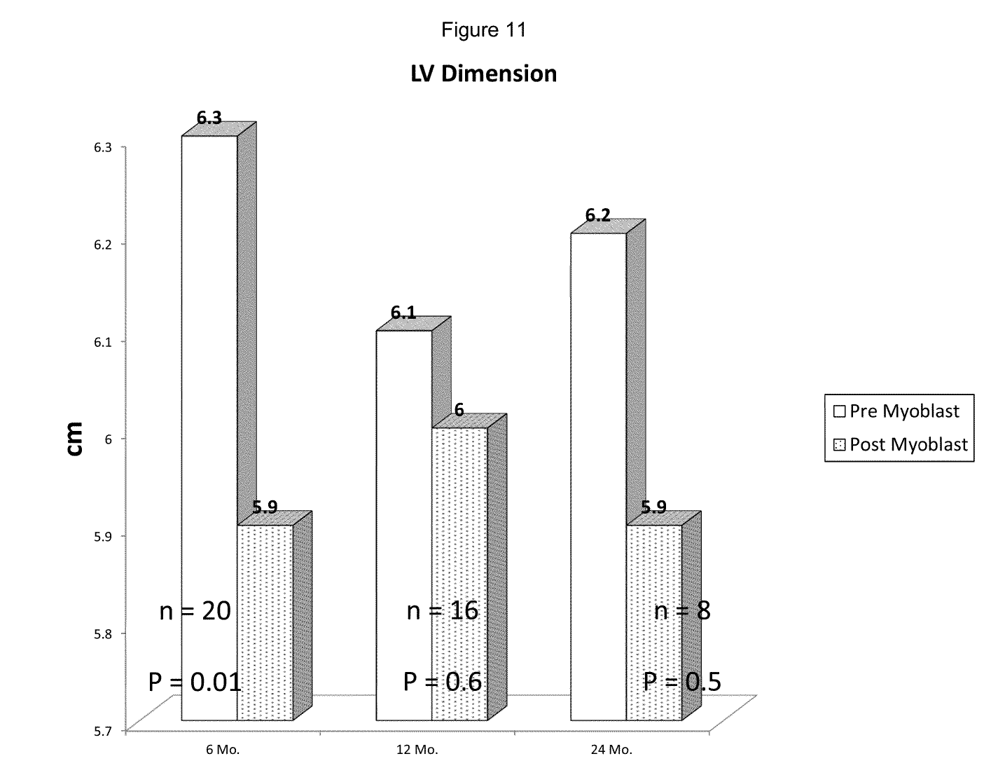

At sacrifice, a consistent trend toward improved cardiac function was seen in the treatment groups relative to controls (Table 6). Given that there were no obvious differences between improvements in cardiac function between animals which were injected with 300 million versus 600 million ASM, the data from both treatment groups were pooled for analysis. By blinded echocardiographic assessment, the treated animals exhibited a 15% improvement in EF by ECHO versus an −10% deterioration in control animals, and a 2% decreases in EF by LV gram versus a 12% deterioration in control animals. Finally, the mean APUPV improved by 23% in treated animals but declined 4% in control animals. Representative examples of the 3-dimensional NOGA unipolar voltage maps at baseline and at the completion of the study are shown in FIG. 5.

Histology.

Histological analysis of sections taken through the anterior left ventricular wall of each treatment pig showed lack of cell survival 60 days after implantation. No injected myoblasts or more mature multinucleated myotubes were detected using H&E and trichrome stains, or myoblast specific myosin heavy-chain immunostaining (MY-32). Also, immuno-staining for nuclear BrdU was negative on all animals. Lesions in graft-recipient pigs were not more severe or qualitatively different than those in the control animals.

### Discussion

Experimentally, myoblasts have been delivered into the injured heart using a number of methods, including intravascular infusion into the coronary circulation (D. A. Taylor et al., Proc. Assoc. Am. Physicians, 1997, 109: 245-253), transvenous delivery (C. Brasselet et al., J. Am. Coll. Cardiol., 2003, 41: 67A-68A), direct epicardial injection into the injured myocardium (D. A. Taylor et al., Nat. Med., 1998, 4: 929-933; M. Jain et al., Circulation, 2001, 103: 1920-1927; S. Ghostine et al., Circulation, 2002, 106(Suppl. I): I131-136; N. Dib et al., Cell Transplant., 2005, 14: 11-19), and most recently, by catheter-based endoventricular delivery (N. Dib et al., J. Endovasc. Ther., 2002, 9: 313-319; B. Chazaud et al., Cardiovasc. Res., 2003, 58: 444-450). Catheter-based delivery is more challenging than direct injection since the myocardial wall is thinner than in healing tissue. Thus, the accuracy, retention, biodistribution and safety of using a needle injection catheter to deliver the cells to a thin wall were examined and the risk of perforation and cell leakage were assessed.

The safety data indicate that percutaneous, catheter-based transplantation of ASM does not have a deleterious effect on the general well-being of the recipient animals or the infarcted swine heart muscle. In addition, the trend toward improved myocardial function seen in the two treatment groups compared to controls not only supports the safety findings, but also indicates that catheter-based delivery is feasible and results in greater overall heart function.

Using percutaneous catheter delivery of iridium labeled myoblasts, the cells were accurately targeted to the infarct zone in the anterior and septal apex of the pig heart. Within 2 hours, 4.6% of the cells were retained in the site of implantation and 5.1% were localized in the lung. Biodistribution to other areas of the heart and the spleen was very low, and no cells were detected in the other analyzed tissues: brain, kidney and liver. In total, only approximately 9% of the cells were detected in the tissues examined, indicating that the remaining cells were distributed in other fluids and tissues. Other short-term retention studies using catheter-based delivery methods which have reported 43% retention of microspheres immediately after injection (P. M. Grossman et al., Cathet. Cardiovasc. Intervent., 2002, 55: 392-397), and 11% retention of myoblasts 2 hrs after injection (C. Brasselet et al., J. Am. Coll. Cardiol., 2003, 41: 67A-68A).

In our safety and feasibility study of 6 animals, there were no complications related to the transplant procedure. Interrogation of a surgically implanted loop recorder revealed that no arrhythmias occurred following endocardial catheter injection of up to 756 million cells in a total volume of 5.9 mL. There were also no elevation of cardiac enzymes at 2 months which might indicate inflammatory or tumorgenicity processes. However, we did observe complications that occurred after MI and prior to the cell transplant procedure; three pigs did not survive the MI, one pig had sustained VT and two had sinus tachycardia.

Albeit with a small sampling size, we observed a trend toward improvement in heart function by ECHO, LV gram and conductive output, despite negative histochemical staining with MY-32. This paradoxical finding suggests that the improvement in the treated arm might be due to transient myoblast cell survival, recruitment of other cell types to the area of myocardial infarction, nascent angiogenesis or prevention of further ischemic damage. Yet, we have no data to support these mechanisms and cannot rule out the possibility that the observed improvements are not significant or reproducible. A larger animal study would be necessary to confirm the reported cardiac changes in this study. It is known from a large number of animal and clinical studies in species other than pigs (e.g., rats (M. Jain et al., Circulation, 2001, 103: 1920-1927), rabbits (D. A. Taylor et al., Nature Med. 198, 4: 929-933), sheep (S. Ghostine et al., Circulation, 2002, 106(Suppl. I): I131-136), and humans (A. A. Hagège et al., Lancet, 2003, 361: 491-492; F. Pagani et al., Circulation, 2002, 106(Suppl. II); II463)) that myoblasts transplanted by epicardial delivery survive and form myotubes and myofibrils, suggesting that grafted myoblasts are able to survive in a foreign environment. We currently speculate that porcine myoblasts have a unique property which does not allow them to survive long-term in the normal or infarcted myocardium. This conclusion is based on unpublished findings in other studies using epicardially injected porcine myoblasts which did not show ASM survival beyond a few days after implantation (data not shown). In the literature, there are references to short term myoblasts transplant studies in the porcine heart (C. Brasselet et al., J. Am. Coll. Cardiol., 2003, 41: 67A-68A; B. Chazaud et al., Cardiovasc. Res., 2003, 58: 444-450), but no long-term studies describing ASM survival.

In summary, our data indicate that delivery of autologous skeletal myoblasts via a percutaneous endoventricular technique into a coil-infarcted swine myocardium may be performed safely, without adverse events related to the procedure or toxicity of the cells. Secondarily, our findings suggest that implantation of ASM via percutaneous catheter may improve cardiac function.

### Example 4

### Safety and Feasibility of Clinical Percutaneous Transplantation of Autologous Skeletal Myoblasts into the Ischemic Myocardium

### Feasibility and Safety of Autologous Myoblast Transplantation During Open-Heart Surgery

Twenty-seven (27) patients with a history of ischemic cardiomyopathy participated in a phase I, non-randomized, multi-center clinical trial of autologous skeletal myoblast transplantation concurrent with coronary artery bypass grafting (CABG) or left ventricular assist device (LVAD) transplantation. Twenty four (24) patients with a history of previous myocardial infarction and a left ventricular ejection fraction less that 30% (12 patients) or less than 40% (12 patients) were enrolled in the CABG study. A second group of 10 patients with an ejection fraction less than 40% was approved and 9 patients were enrolled in the LVAD study. The average age of CABG and LVAD subjects was 55.2±10.7 years and 56±8.3 years, respectively.

In the LVAD study, six (6) patients underwent LVAD implantation as a bridge to heart transplantation and donated their heart for testing at the time of heart transplant. A skeletal muscle biopsy of approximately 2-5 grams was excised from the biceps or quadriceps of each patient. Myoblasts were isolated and expanded over a period of 2-3 weeks. Between 3 and 30 direct injections of myoblasts were delivered into the area of infarction at the time of surgery using one of four escalating cell doses ranging from 2.2×10⁶ cell to 300×10⁶ cells. Myoblasts were delivered successfully in all subjects without any injection-related complications. Purity of the myoblasts was 43% and 98% based on flow cytometry analysis for CD56.

Follow-up examinations as long as 24 months (mean=17 months) revealed no adverse events associated with the cells nor the injection procedure. There were two deaths, one in the LVAD group due to line sepsis 3 months post-transplantation, and one in the CABG group 12 days post-procedure due to myocardial infarction, as confirmed by autopsy. Two patients experienced episodes of non-sustained ventricular tachycardia that was considered possibly related to the myoblast transplantation. These events occurred early in the post-operative period (7 and 10 days), one which was symptomatic and one non-symptomatic. Both underwent ICD implantation and no further events have been observed. A third patient also experienced non-sustained ventricular tachycardia one week post-transplant. This was not considered to be related to the cell transplant due to findings of significant stenosis in the left internal mammary artery graft, which was successfully treated with beta blockers. No further arrhythmias were observed up to one year after placement of an ICD. Echocardiography and magnetic resonance imaging revealed thickening of the scar region. Positron emission tomography revealed viable tissue in the area of injection. The potential to regenerate functioning muscle using autologous myoblast transplantation could have significant therapeutic application after acute myocardial infarction.

### Feasibility and Safety of Percutaneous Transplantation of Autologous Skeletal Myoblasts

Study Objectives:

This is a phase I, prospective, open-label, randomized, clinical study to evaluate the tolerability and feasibility of autologous cultured skeletal myoblast transplantation versus maximal medical therapy in patients with congestive heart failure (NYHA Class II and IV, see Table 7). This study enrolls 24 patients having a diagnosis of previous myocardial infarction and an ejection fraction <40%, secondary to previous myocardial infarction.

TABLE 7
New York Heart Association (NYHA) Functional Classification

| | |
|---|---|
| Class I | No limitation of physical activity; no symptoms with |
| | ordinary physical activity |
| Class II | Slight limitation of physical activity; comfortable at rest; |
| | symptoms with ordinary physical activity |
| Class III | Marked limitation of physical activity; comfortable at rest; |
| | symptoms with less than ordinary physical activity |
| Class IV | Unable to carry on physical activity without discomfort; |
| | symptoms at rest |

After reading and signing an informed consent, patients underwent screening and baseline evaluations. At this point, each patient was prescribed maximal medical therapy for 2 months. At the end of the two-month period, if the patient was determined to still be in Class II-IV, the patient was then randomized. Patients were then assigned to a treatment group: Group 1, which corresponds to Autologous Myoblast Transplantation or Group 2, which corresponds to medical therapy only.

Group 1 patients underwent a muscle biopsy taken under local anesthesia from the patient's quadriceps muscle. The muscle biopsy was placed in biopsy medium and transported to the cell culture laboratory for cell expansion. Skeletal myoblasts were expanded over a period of from 4-6 weeks. Autologous cultures skeletal myoblasts were transplanted into the endocardial surface at the site of previous myocardial infarction. The site of myocardial infarction was identified using electromechanical mapping. Myoblast cell dosage starting at 10×10⁶ cells up to 300×10⁶ cells at a concentration of approximately 100×10⁶ cells per mL were injected. A maximum of 30×10⁶ cells was injected in the first three patients. Patients 4, 5, and 6 received up to 100×10⁶ cells; patients 7, 8, and 9 received up to 300×10⁶ cells and patients 10, 11 and 12 received up to 600×10⁶ cells. The other 12 patients (13 to 24) received up to 600×10⁶ cells. Injections were made approximately 1 cm apart into the area of infarct. Patients were monitored throughout the transplantation procedure. Patients were hospitalized for a minimum of 24 hours and managed according to the current standard of care until recovered from catheterization. Patients were assessed at 7 days, 2 weeks, 1, 3, and 6 months after transplantation. A long-term follow-up visit was performed at 1 year. Safety evaluations and cardiac function evaluations were performed at each of these visits.

### Prior Medications

Patients received treatment with maximal medical therapy for at least 2 months prior to cell transplantation. Maximal medical therapy includes the following medications (unless hemodynamic parameters or intolerance contraindicate their use): diuretic, angiotensin II converting enzyme (ACE) inhibitor (or, if intolerant to ACE inhibitors, angiotensin II antagonist), digoxin, carvedilol, and platelet aggregation inhibitors (e.g., aspirin, ticlopidine, or clopidogrel). Maximum medical therapy was reviewed and adjusted as necessary and the patient was maintained for 2 months on this regimen prior to randomization. If the patient was still in class II-IV, the patient was randomized. A patient may undergo the biopsy during screening with the knowledge that if their condition improves to class I or less during the 2 month surveillance they will be excluded from the study.

Baseline and Screening Evaluations:

The following baseline clinical evaluations were performed on each patient as described below: (1) History and physical exam, including vital signs (blood pressure, heart rate and oral temperature); (2) Minnesota Living with Heart Failure Questionnaire and 6-Minute Walk test; (3) Laboratory tests, as described in Table 8; (4) Chest X-ray (within previous 6 months); (5) 12-Lead electrocardiogram; (6) Echocardiogram performed under optimal medical therapy; (7) Holter monitor (48 hours); (8) Stress Nuclear/Viability Assessment performed while the patient is under optimal medical therapy; (9) Left heart catheterization (within previous 6 months); (10) T-wave alternant test; (11) Review of eligibility checklist criteria; and (12) NYHA confirmed by Medical Monitor.

TABLE 8
Description of Clinical Laboratory Tests

| Test | Description |
|---|---|
| CBC | Complete blood count: hemoglobin, hemotocrit, |
| | platelets, white blood cell count, differential, |
| | red cell indices |
| Chemistry | Sodium, potassium, chloride, total CO₂, |
| | glucose, blood urea nitrogen (BUN), creatine, alanine |
| | amino transferase (ALT), aspartate amino transferase |
| | (AST), bilirubin, calcium, total protein, albumin, |
| | alkaline phosphatase, uric acid, phosphorus |
| Hepatitis | B surface antigen, C |
| HIV | Antibodies against human immune deficiency virus 1 |
| RPR | Syphyllis |
| CMV | Cytomegalovirus IgG/IgM |
| PT/PTT | Prothrombin time, partial prothrombin time |
| ABO Rh profile | Blood typing |
| Pregnancy | Serum for women with childbearing potential |
| Cardiac enzymes | Total creatine phosphokinase (CPK), |
| | creatine phosphokinase-myocardial band (CPK-MB), |
| | troponin T |
| Urinalysis | General urine (appearance, specific gravity, pH, |
| | protein, glucose, ketones, bilirubin, hemoglobin, |
| | number and type of cells, characterization of |
| | sediment) and protein/creatine ratio |
| BNP | B-type Natriuretic Peptide |

Loop Recorder Implantation:

Once the patient has been enrolled into the study, an outpatient visit was scheduled for implantation of an Insertable Loop Recorder (ILP), Medtronic REVEAL PLUS Model 9526. As already mentioned above, this is a single-use programmable device containing two electrodes on the body of the device for continuous (i.e., looping) recording of a subcutaneous electrocardiogram during arrhythmic events. In a single-incision procedure, an approximate 2 cm incision was made. The device was placed in a subcutaneous pocket approximately 4 cm×2 cm. The wound was closed in a conventional manner. The patient was instructed to activate the device in the event that they experience dizziness, light-headedness, chest discomfort, shortness of breath, palpitation, or any unusual felling. The device can be interrogated during follow-up visits or as needed. The electrocardiogram recordings can be evaluated for occurrence of arrhythmias.

Skeletal Muscle Biopsy:

An outpatient visit was scheduled for the muscle biopsy (the outpatient loop recorder implant and muscle biopsy procedures may be performed during the same visit). Approximately 5 grams of skeletal muscle, taken from the patient's quadriceps, was obtained under local anesthesia. An incision, approximately 5 cm long, was made longitudinally along the anterolateral aspect of the thigh in the center of the thigh. Dissection was carried through the soft tissue and fascia and the rectus femoralis was identified and exposed. The incision was repaired in layers. The specimen was placed in biopsy medium and sent to the cell culture facility.

If the patient was unable to undergo the myoblast transplantation procedure due to illness, change in health condition or other unforeseen circumstances, the cell specimen was destroyed. A second biopsy was required, if the patient continued to participate in the study.

Culture of Autologous Skeletal Myoblasts:

Autologous cultured skeletal myoblasts were isolated through a series of steps, involving mechanical dissection, washing, and resuspension. The cells were expanded over a period of from 4-6 in a culture facility.

Myoblast Transplantation:

Between 4-6 weeks after the muscle biopsy, the patients were scheduled to undergo intra-myocardial injections of the autologous myoblasts. Patients continued all heart failure medications at their current prescribed dosages.

Standard procedures for right and left cardiac catheterizations were followed. Patients were not allowed to take anything by mouth after midnight of the night before the transplantation procedure. Beginning and ending times for the whole procedure and times for all intermediary procedures were noted.

Patients received a heparin bolus (5,000 units to maintain activated clotting time values [ACT]). During the procedure, patients were constantly monitored for: blood pressure, heart rate, oxygen saturation, 2-lead electrocardiogram, respiration rate, and coagulation [ACT].

More specifically, in the case of a right heart catheterization, an 8-french sheath was introduced to the femoral vein after puncturing the femoral vein with modified Seldinger technique, a Swan Ganz catheter was introduced to the right heart and pressure was obtained from the right atrium, right ventricle, pulmonary artery and wedge pressure. Cardiac output was measured three times. Right atrium and pulmonary artery saturations were also measured. In the case of a left heart catheterization, an 8-french sheath was introduced into the femoral artery. A JL 4 or appropriate diagnostic catheter was used to visualize the left coronary arteries and JR 4 or appropriate diagnostic catheter was used to visualize the right coronary artery. A pigtail catheter was used for left ventriculography. Appropriate catheters were used for the grafts.

Electromechanical Mapping Study:

Coronary angiography was performed after administering intracoronary nitroglycerin. All patients had left ventriculography in both left anterior oblique (LAO) and right anterior oblique (RAO) view. The location of the infarcted tissue was identified using Biosense NOGA™ mapping following left heart catheterization.

The electromechanical mapping system used comprises: a location pad containing three coils generating ultraslow magnetic field energy, a stationary reference catheter with a miniature magnetic field sensor located on the body surface, a navigation sensor mapping catheter (7F) with deflectable-tip and electrodes providing endocardial signal including voltage and contractility, and a workstation for information processing and 3-dimensional left ventricle reconstruction. The map obtained included voltage map and local shortening map; areas of normal tissue, ischemia and infarction were identified; a number and color scale were assigned by the computer indicating the voltage in each area of the myocardium. Other number and color scales were assigned for each area on the local shortening map indicating the contractility of that segment.

More specifically, an adhesive reference patch was placed on the back of the patient, to the left of the spine at T7 level. Under fluoroscopic guidance to the descending thoracic aorta, the NOGA™ mapping catheter was deflected to form a J shape, and was introduced across the aortic valve into the left ventricle. The location of the mapping catheter was gated to the end diastole and recorded relative to the location of the fixed reference catheter at that time, thus compensating for subject or cardiac motion. As the catheter tip was moved over the left ventricle endocardial surface, the system continuously analyzed its location in 3-dimensional space without the use of fluoroscopy.

Results were collected from unipolar (UP) and bipolar (BP) simultaneous recording filtered at 0.5 to 400 Hz. The stability of the catheter-to-wall contact was evaluated at every site in real time. Points were deleted from the map if one of the following criteria was met: (1) a premature beat or a beat after a premature beat; (2) location stability, defined as a difference of >5 mm in end-diastolic location of the catheter at 2 sequential heartbeats; (3) loop stability, defined as an average distance of >5 mm between the location of the catheter at 2 consecutive beats at corresponding time intervals in the cardiac cycle; (4) cycle length that deviated >10% from the median cycle length; (5) different morphologies of the local electrocardiogram at 2 consecutive beats; (6) local activation time differences of >5 ms between 2 consecutive beats; and (7) different QRS morphologies of the body surface electrocardiogram.

By setting a “triangle fill threshold” value, the operator could choose the minimum triangle size for which the program closes a face on the reconstructed chamber. This feature allowed the operator to determine the degree to which the system interpolates between actual data points and ensures that a minimal point density is met at each mapped region. All maps were acquired with an interpolation threshold of 15 nm between adjacent points. The 3-dimensional left ventricle endocardial reconstruction was updated in real time with the acquisition of each new site and displayed continuously on a Silicon Graphics workstation.

Intra-Myocardial Injection Procedure:

Myoblasts were injected into the endoventricular surface of the infarction area using the Biosense intra-myocardial injection catheter. Doses were escalated with a starting dose of 10×10⁶ cells followed by 30×10⁶ cells, 100×10⁶ cells, and 300×10⁶ cells. Each group included 3 patients, except the last one (300×10⁶ cells) which included another 12 patients. The cells were concentrated at 100×10⁶ per mL. The injections were made approximately 1 cm apart into the area of infarction at a volume of 0.1 mL (10 million cells) in the 30 million dose group and 0.25 mL (25 million cells) in the rest of the groups.

In each case, an introducer sheath of at least 8F was inserted into the right or left femoral artery using standard procedures for percutaneous coronary angioplasty. Following insertion of the arterial sheath, heparin and supplement were administered as needed to maintain an ACT (activated clotting time) of 200-250 seconds throughout the interventional portion of the procedure.

After orientation of the injector catheter to the treatment zone (i.e., infarcted area of the heart muscle), using the baseline Biosense NOGA™ electro-mechanical map and fluoroscopic guidance when necessary the operator established the stability of the injection catheter on the endocardial surface (based on the recording of loop-stability value <4 and cycle length stability during sinus rhythm). Then the injection needle was extended into the myocardium to a depth of approximately 60% of the scar thickness as measured by echocardiography to avoid risk of perforation (a myocardial scar thickness below 5 mm was excluded). Injections were administered in a volume of 0.25 mL or less. Ten to twenty-five million cells per injection site were spaced 1 cm apart, into the center and around the area of the infarct. The density of injection sites depended upon individual patient left ventricle endocardial anatomy and the ability to achieve a stable position on the endocardial surface without catheter displacement or PVCs. The workstation software provided precise annotation of the location in 3-dimensional (3-D) space for each injection site. After the conclusion of the endocardial injection portion of the procedure, the injection catheter was removed.

During the transplantation procedure, all vital signs were constantly monitored for evidence of serious complications, especially arrhythmias, perforation, bradycardia, or tachycardia. The procedure was prematurely terminated for a variety of reasons, such as (1) technical device malfunctions (e.g., inability to accurately sense the NOGA™ catheter location or failure to inject the myoblasts due to device or catheter malfunction); (2) operator failures (e.g., catheter or operator inability to achieve a sufficiently stable endocardial position to perform the injection procedure); (3) complications (serious ventricular arrhythmias requiring repetitive electrical cardioconversion; severe vascular injury during insertion of the Biosense catheter; catheter trauma to the coronaries due to inadvertent placement of the NOGA-Star injection catheter or injector into the coronary ostium which may result in dissection, abrupt closure, perforation, or severe ischemia; trauma to the aortic valve causing hemodynamic compromise associated with acute aortic regurgitation; perforation or trauma to the mitral valve apparatus due to placement of the NOGA-Star catheter or injector or due to needle puncture; LV perforation due to catheter placement or needle penetration into the pericardial space.

Post-Transplantation Evaluation:

Following completion of the transplantation procedure, the patient was monitored in the catheter laboratory for 10 minutes. An electrocardiograph and analysis of cardiac enzymes were performed, and the patients was then admitted to the cardiac telemetry unit until discharge. Heart rate, blood pressure, pulse oximetry, and distal pulses were monitored every 15 minutes for one hour, every 30 minutes for 2 hours, every hour for 4 hours, and every 4 hours until discharge. Electrocardiograph and analysis of cardiac enzymes were performed at approximately 8 and 16 hours following the procedure. Within approximately 24 hours, the patient underwent several tests including cardiovascular examination, CBS, cardiac enzymes, echocardiograph, electrocardiograph and chest X-ray. Patients were discharged home approximately 24 hours following a satisfactory examination. The ILR was interrogated prior to discharge. Follow-up visits were scheduled at 2-days, 7 days, 2 weeks, 1 month, 3 months, 6 months, and 12 months post discharge.

Data Evaluation:

The primary objective of this study was to evaluate the tolerability and feasibility of percutaneous delivery of autologous cultured skeletal myoblasts in patients with congestive heart failure.

The tolerability was evaluated based on the number of patients without the following serious adverse events: (1) cerebrovascular incident (stroke); (2) ventricular tachycardia or fibrillation causing cardiac arrest; (3) ventricular perforation as demonstrated by tachycardia, systolic arterial blood pressure <70 mm Hg, and pericardial effusion; (4) infection and/or sepsis determined to be related to the myoblast transplantation; (5) creatine phosphokinase and MB levels greater than 3 times the normal limit at 2 weeks that are determined to be related to the myoblast transplantation; and (6) death within one month of procedure. If two patients experience any one of the following serious adverse events, which is considered related to the cell transplant, the study was to be stopped. This number was selected because it would represent a greater number than expected with normal catheterization procedures. The tolerability of the myoblast transplantation preparation was evaluated based on the number of patients not experiencing reactions to the preparations. The patient could experience an allergic reaction associated with components of the myoblast preparation or infection caused by contamination of the cell preparation. Any potential reactions was noted by monitoring heart rate, blood pressure, and temperature.

The feasibility of the myoblast transplantation procedure was evaluated based on the number of patients with successfully completed myoblast transplantation. A successfully completed transplant patient was defined as a patient who completed the procedure with no life-threatening complications. Patients must have received at least ⅔ of the calculated dose.

To determine improvement in cardiac function, the post-transplantation assessments were compared to the baseline assessments. At 1 week, 2 weeks, 1 month, 3 months, and 6 months after treatment, patients underwent echocardiography assessment of the left ventricular function, wall motion and thickness, and valve function. Three months after treatment, patients underwent: (1) left and right heart catheterization to assess the left ventricular function, wall motions, left and right heart pressures, and cardiac output; (2) Stress Nuclear/Viability Assessment to assess change in size of infarction; and (3) NOGA mapping to assess the voltage (size of infarction) and local shortening. The changes from baseline to month 1, 3 and 6 were summarized for regional left ventricular wall function in engrafted areas. Voltage and local shortening of all cardiac segments on the NOGA map obtained at baseline and 3 months were compared. A positive improvement in cardiac function was considered: a mean increase in wall thickness of 2-3 mm, or a mean increase in ejection fraction of >5%. Changes in quality of life assessment (MLHFQ) and 6-Minute Walk test from baseline to 3, 6, and 12 months follow-up were summarized.

### CABG and Cell Transplantation Group—Results

The cumulative patient enrollment in the CABG and Cell Transplantation Group is shown in FIG. 8. Table 9 presents the baseline demographics for patients in this Group. The viability of cell injected was between 85% and 98% and cell purity was between 47% and 98% (see FIG. 7). Cell delivery was 100% successful without injection-related complications. Adverse events observed are listed in Table 11. These events were determined to be unrelated to transplantation by the Data Safety Monitoring Board (DSMB). Other results obtained in this Group, including NYHA Class, electrocardiogram, LV diastolic volume and LV dimension, are shown in FIG. 8, FIG. 9, FIG. 10 and FIG. 11, respectively.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the claims.

1. A method for treating a dysfunctional heart comprising steps of: identifying a subject in need of treatment for cardiac dysfunction; and delivering a cell composition comprising skeletal myoblasts to the subject's dysfunctional heart using a catheter-based system, wherein at least part of the catheter-based system is inserted into a blood vessel of the subject.

2-25. (canceled)

26. A method for treating a dysfunction heart comprising steps of: identifying a subject in need of treatment for cardiac dysfunction; and delivering a cell composition comprising skeletal myoblasts to the subject's dysfunctional heart using a catheter-based system, wherein at least part of the catheter-based system is inserted into a blood vessel of the subject, and wherein the cell composition is delivered in conjunction with an open-chest procedure.

27-53. (canceled)

54. A method for treating heart tissue of a patient by catheter delivery of a suspension of cells, comprising the steps of: electromechanical mapping endocardial surfaces of the heart of the patient; identifying, from the electromechanical mapping, sites along for endocardial surface for injection of the suspension of cells; injecting the suspensions of cells into the identified sites on the endocardial surface using an endocardial catheter injector.

55. The method of claim 54, wherein the endocardial catheter injector is part of a catheter-based system comprising a cardiac mapping system that is equipped with at least one mapping electrode and that is adapted for the step of electromechanical mapping of the endocardial surfaces.

56. The method of claim 55, wherein the endocardial catheter injector comprises at least one needle that is adapted to inject the suspension of cells into a localized region of the patient's heart.

57. The method of claim 54, which results in one or more of: reduction of the severity of cardiac dysfunction, improved cardiac function, at least partial restoration of structural integrity of injured myocardium, at least partial restoration of functional integrity of injured myocardium, improved cardiac systolic function, improved cardiac diastolic function, improved cardiac muscle elasticity, improved cardiac muscle contractility, and increased left ventricular function.

58. The method of claim 54, wherein said method is used to treat or repair a myocardial infraction.

59. The method of claim 54, wherein said method is used to improve heart function in coronary heart disease.

60. The method of claim 54, wherein suspension of cells include myoblast cells isolated and expanded in vitro from muscle from said patient.

61. The method of claim 54, wherein suspension of cells include stem cells isolated and expanded in vitro from bone marrow from said patient.

62. The method of claim 61, wherein said stem cells isolated and expanded from bone marrow from said patient include mesenchymal stem cells, hematopoietic stem cells, or a mixture of both.

63. The method of claim 54, wherein the electromechanical mapping and endocardial catheter injector are part of an integrated injection catheter, wherein the catheter is a multi-electrode, percutaneous catheter with a deflectable tip and injection needle designed to inject agents into the myocardium, the tip of the injection catheter being equipped with a location sensor and the injection needle is a retractable, hollow needle for fluid delivery.
